(19) United States
(12) Patent Application Publication
(10) Pub. No.: US 2023/0098151 A1
(43) Pub. Date: Mar. 30, 2023

(54) TESTING FOR VIRUSES AND CELLULAR BIOMARKERS

(71) Applicant: HERO SCIENTIFIC LTD., Jerusalem (IL)

(72) Inventors: Lazar FRUCHTER, Efrat (IL); Leah FORGOSH, West Hartford, CT (US); Boaz ARIELI, Mevaseret Tzion (IL); Robert Eric LEVITZ, Beit Shemesh (IL)

(73) Assignee: HERO SCIENTIFIC LTD., Jerusalem (IL)

(21) Appl. No.: 17/801,452

(22) PCT Filed: Mar. 11, 2021
(86) PCT No.: PCT/IB2021/052055

§ 371 Date: Aug. 22, 2022

Related U.S. Application Data
(60) Provisional application No. 62/988,259, filed on Mar. 11, 2020.

(51) Int. Cl. G01N 33/569; C12N 15/10

(52) U.S. Cl. CPC G01N 33/569; C12N 15/1017

(57) ABSTRACT

A method is provided for testing a non-centrifuged oronasopharyngeal fluid sample (22) taken from a human or non-human animal for the presence of a virus, the method including passing the non-centrifuged oronasopharyngeal fluid sample (22) through one or more porous filters (32, 1932) to separate epithelial cells from the non-centrifuged oronasopharyngeal fluid sample by size-based filtration. Thereafter, an extraction liquid is prepared by extracting cellular components of the epithelial cells separated from the non-centrifuged oronasopharyngeal fluid sample (22). Thereafter, the extraction liquid is tested for the presence of the virus. Other embodiments are also described.

### CROSS-REFERENCE TO RELATED APPLICATIONS

The present application claims priority from U.S. Provisional Application 62/988,259, filed Mar. 11, 2020, which is assigned to the assignee of the present application and is incorporated herein by reference.

### FIELD OF THE APPLICATION

Applications of the present invention relate to testing for the presence of particulates, such as viruses and cellular biomarkers, in fluids.

### BACKGROUND OF THE APPLICATION

Many techniques exist for testing for the presence of viruses and cellular biomarkers for aiding in disease diagnosis. For example, testing for Influenza virus and for Severe Acute Respiratory Syndrome Coronavirus 2 (SARS-CoV-2) includes molecular-based detection methods and immunoassay detection methods. Influenza virus testing includes the testing of nasal swabs, nasopharyngeal swabs, nasal aspirates, nasopharyngeal aspirates, nasal washes, nasopharyngeal washes, throat swabs, and a combination of samples. SARS-CoV-2 testing includes the testing of nasal swabs, nasopharyngeal swabs, nasal aspirates, nasopharyngeal aspirates, nasal washes, nasopharyngeal washes, throat swabs, gargle fluids, saliva samples, and a combination of samples.

Testing for cellular biomarkers also includes molecular-based detection methods and immunoassay detection methods. Cellular biomarker testing includes the testing of saliva, blood, urine, and feces. Cellular biomarker testing includes testing to detect cellular products, such as proteins, antibodies, antigens, enzymes, peptides, nucleic acids, carbohydrates, hormones, lipids, substrates, and metabolites, which act as circumstantial evidence that a specific cell or a specific cell type is present in a sample and/or in a human or non-human animal from which the sample was taken. Cellular biomarkers may be tumor-specific cellular biomarkers, inflammatory-specific cellular biomarkers, and/or disease-specific cellular biomarkers.

Some cellular biomarkers are tumor-specific cellular biomarkers, such as cellular products of tumor cells, that act as circumstantial evidence that a tumor cell may be present or may be present in higher-than-normal levels in a sample and/or in a human or non-human animal from which the sample was taken. For example, some tumor-specific cellular biomarkers that may be present or may be present in higher-than-normal levels in oral fluid samples, such as high levels of interleukin-8 mRNA and/or interleukin-8 protein, act as circumstantial evidence that cancerous cells, such as oral cancer cells, may be present in the sample and/or in a human or non-human animal from which the sample was taken.

Some cellular biomarkers are inflammatory-specific cellular biomarkers, such as cellular products of cells involved in the inflammatory response, that act as circumstantial evidence that a specific cell or cell type involved in the inflammatory response may be present or may be present in higher-than-normal levels in a sample and/or in a human or non-human animal from which the sample was taken. For example, some inflammatory-specific cellular biomarkers, such as CD64, that act as circumstantial evidence that a specific cell or cell type involved in the inflammatory response, such as leukocytes, may be present or may be present in higher-than-normal levels in a sample and/or in a human or non-human animal from which the sample was taken, indicate that the human or non-human animal from which the sample was taken may have an infection.

Some cellular biomarkers are disease-specific cell markers, such as cellular products of cells with a disease state, that act as circumstantial evidence that cells with a specific disease state may be present or may be present in higher-than-normal levels in the sample and/or in a human or non-human animal from which the sample was taken, For example, some disease-specific cellular biomarkers, such as HIV-specific antibodies in saliva, that act as circumstantial evidence that viral infected cells, such as HIV-infected cells, may be present in the sample and/or in a human or non-human animal from which the sample was taken, indicate that the human or non-human animal from which the sample was taken may have a disease caused by a viral infection, such as AIDS.

### SUMMARY OF THE APPLICATION

Some embodiments of the present invention provide devices and methods for testing a non-centrifuged oronasopharyngeal fluid sample taken from a body of a patient for the presence of a virus. For some applications, a method is provided that comprises passing the non-centrifuged oronasopharyngeal fluid sample through a porous filter to separate epithelial cells from the non-centrifuged oronasopharyngeal fluid sample by size-based filtration. Thereafter, an extraction liquid is prepared by extracting cellular components of the epithelial cells separated from the non-centrifuged oronasopharyngeal fluid sample. Thereafter, the extraction liquid is tested for the presence of the virus. For some applications, the epithelial cells include one or more types of cell selected from the group consisting of: squamous cells, columnar cells (e.g., goblet cells), and cuboidal cells.

In some applications, the non-centrifuged oronasopharyngeal fluid sample is passed through two porous filters, one of which has a pore size selected to generally prevent passage of the epithelial cells, and the other of which has a pore size selected to generally prevent passage of free viral particles.

Some embodiments of the present invention provide devices and methods for testing a non-centrifuged oronasopharyngeal fluid sample taken from a body of a patient for the presence of a cellular biomarker. For some applications, a method is provided that comprises passing the non-centrifuged oronasopharyngeal fluid sample through a porous filter to separate animal cells from the non-centrifuged oronasopharyngeal fluid sample by size-based filtration. Thereafter, an extraction liquid is prepared by extracting cellular components of the animal cells separated from the non-centrifuged oronasopharyngeal fluid sample. Thereafter, the extraction liquid is tested for the presence of the cellular biomarker.

For some applications, the animal cells include one or more types of cell selected from the group consisting of: epithelial cells (squamous cells, columnar cells (e.g., goblet cells), or cuboidal cells) and leukocytes (eosinophils, neutrophils, lymphocytes, monocytes, mast cells, basophils, macrophages, or histiocytes).

There is therefore provided, in accordance with an Inventive Concept 1 of the present invention, a method for testing a non-centrifuged oronasopharyngeal fluid sample taken from a human or non-human animal for the presence of a virus, the method including:

passing the non-centrifuged oronasopharyngeal fluid sample through one or more porous filters to separate epithelial cells from the non-centrifuged oronasopharyngeal fluid sample by size-based filtration;

thereafter, preparing an extraction liquid by extracting cellular components of the epithelial cells separated from the non-centrifuged oronasopharyngeal fluid sample; and thereafter, testing the extraction liquid for the presence of the virus.
Inventive Concept 2. The method according to Inventive Concept 1, wherein testing the extraction liquid includes testing the extraction liquid while the extraction liquid is in contact with at least one of the one or more porous filters.
Inventive Concept 3. The method according to Inventive Concept 1, wherein testing the extraction liquid for the presence of the virus includes removing at least a portion of the extraction liquid from contact with the one or more porous filters, and testing at least a portion of the removed extraction liquid.
Inventive Concept 4. The method according to Inventive Concept 1, wherein testing the extraction liquid for the presence of the virus includes applying a transport liquid to at least one of the one or more porous filters to produce a mixture of the extraction liquid and the transport liquid, removing a portion of the mixture from the at least one of the one or more porous filters, and testing the removed mixture for the presence of the virus.
Inventive Concept 5. The method according to Inventive Concept 1, wherein preparing the extraction liquid includes extracting, while the epithelial cells are in contact with at least one of the one or more porous filters, the cellular components of the epithelial cells separated from the non-centrifuged oronasopharyngeal fluid sample.
Inventive Concept 6. The method according to Inventive Concept 1, wherein testing for presence of the virus includes quantifying a level of the virus.
Inventive Concept 7. The method according to Inventive Concept 1, wherein filtering the non-centrifuged oronasopharyngeal fluid sample includes filtering between 0.5 and 50 cc of the non-centrifuged oronasopharyngeal fluid sample.
Inventive Concept 8. The method according to Inventive Concept 7, wherein filtering the non-centrifuged oronasopharyngeal fluid sample includes filtering between 4 and 25 cc of the non-centrifuged oronasopharyngeal fluid sample.
Inventive Concept 9. The method according to Inventive Concept 1, wherein the non-centrifuged oronasopharyngeal fluid sample is also non-cultured, and wherein passing the non-centrifuged oronasopharyngeal fluid sample through the one or more porous filters includes passing the non-centrifuged and non-cultured oronasopharyngeal fluid sample through the one or more porous filters.
Inventive Concept 10. The method according to Inventive Concept 1, wherein at least one of the one or more porous filters has an average absolute pore size of between 0.01 and 20 microns.
Inventive Concept 11. The method according to Inventive Concept 10, wherein the average absolute pore size is between 0.1 and 20 microns.
Inventive Concept 12. The method according to Inventive Concept 10, wherein the average absolute pore size is between 0.45 and 9.0 microns.
Inventive Concept 13. The method according to Inventive Concept 10, wherein the average absolute pore size is between 1.2 and 20 microns.
Inventive Concept 14. The method according to Inventive Concept 13, wherein the average absolute pore size is between 2.0 and 20 microns.
Inventive Concept 15. The method according to Inventive Concept 14, wherein the average absolute pore size is between 5.0 and 20 microns.
Inventive Concept 16. The method according to Inventive Concept 12, wherein the average absolute pore size is between 0.45 and 2.0 microns.
Inventive Concept 17. The method according to Inventive Concept 1, wherein at least one of the one or more porous filters has an average nominal pore size of between 0.01 and 20 microns.
Inventive Concept 18. The method according to Inventive Concept 17, wherein the average nominal pore size is between 0.1 and 20 microns.
Inventive Concept 19. The method according to Inventive Concept 18, wherein the average nominal pore size is between 0.45 and 9.0 microns.
Inventive Concept 20. The method according to Inventive Concept 17, wherein the average nominal pore size is between 1.2 and 20 microns.
Inventive Concept 21. The method according to Inventive Concept 20, wherein the average nominal pore size is between 1.2 and 20 microns.
Inventive Concept 22. The method according to Inventive Concept 21, wherein the average nominal pore size is between 5.0 and 20 microns.
Inventive Concept 23. The method according to Inventive Concept 19, wherein the average nominal pore size is between 0.45 and 2.0 microns.
Inventive Concept 24. The method according to Inventive Concept 1, wherein extracting the cellular components of the epithelial cells includes lysing the epithelial cells.
Inventive Concept 25. The method according to Inventive Concept 1, further including, after passing the non-centrifuged oronasopharyngeal fluid sample through the one or more porous filters and before preparing the extraction liquid, adding, to at least one of the one or more porous filters, material from a nasal swab or a nasopharyngeal swab of the human or non-human animal.
Inventive Concept 26. The method according to any one of Inventive Concepts 1-25,

wherein preparing the extraction liquid includes extracting and exposing a viral target, and

wherein testing the extraction liquid for the presence of the virus includes testing the extraction liquid for the presence of the viral target.
Inventive Concept 27. The method according to Inventive Concept 26,

wherein extracting and exposing the viral target includes extracting and exposing a viral antigen, and

wherein testing the extraction liquid for the presence of the viral target includes testing the extraction liquid for the presence of the viral antigen.
Inventive Concept 28. The method according to Inventive Concept 26,

wherein extracting and exposing the viral target includes extracting and exposing a viral nucleic acid, and

wherein testing the extraction liquid for the presence of the viral target includes testing the extraction liquid for the presence of the viral nucleic acid.
Inventive Concept 29. The method according to Inventive Concept 26,

wherein extracting and exposing the viral target includes extracting and exposing a viral nucleoprotein, and

wherein testing the extraction liquid for the presence of the viral target includes testing the extraction liquid for the presence of the viral nucleoprotein.
Inventive Concept 30. The method according to any one of Inventive Concepts 1-25,

wherein passing the non-centrifuged oronasopharyngeal fluid sample through the one or more porous filters to separate the epithelial cells from the non-centrifuged oronasopharyngeal fluid sample includes passing the non-centrifuged oronasopharyngeal fluid sample through the one or more porous filters to separate the epithelial cells and free viral particles from the non-centrifuged oronasopharyngeal fluid, and

wherein preparing the extraction liquid by extracting cellular components of the epithelial cells includes extracting and exposing a viral target from the cellular components of the epithelial cells and from the free viral particles.
Inventive Concept 31. The method according to any one of Inventive Concepts 1-14, wherein the non-centrifuged oronasopharyngeal fluid sample taken from the human or non-human animal is one or more non-centrifuged oronasopharyngeal fluid samples taken from one or more of oronasopharyngeal cavities of the human or non-human animal.
Inventive Concept 32. The method according to Inventive Concept 31, wherein the non-centrifuged oronasopharyngeal fluid sample includes a non-centrifuged nasal wash taken from the human or non-human animal.
Inventive Concept 33. The method according to Inventive Concept 31, wherein the non-centrifuged oronasopharyngeal fluid sample includes non-centrifuged nasal aspirate taken from the human or non-human animal.
Inventive Concept 34. The method according to Inventive Concept 31, wherein the non-centrifuged oronasopharyngeal fluid sample includes non-centrifuged gargled fluid taken from the human or non-human animal.
Inventive Concept 35. The method according to Inventive Concept 31, wherein the non-centrifuged oronasopharyngeal fluid sample includes a non-centrifuged oral wash taken from the human or non-human animal.
Inventive Concept 36. The method according to Inventive Concept 31, wherein the non-centrifuged oronasopharyngeal fluid sample includes saliva taken from the human or non-human animal.
Inventive Concept 37. The method according to Inventive Concept 31, wherein the non-centrifuged oronasopharyngeal fluid sample includes (a) material from (i) a nasal swab or (ii) a nasopharyngeal swab mixed with (b) (i) non-centrifuged gargled fluid taken from the human or non-human animal or (ii) nasal wash taken from the human or non-human animal.
Inventive Concept 38. The method according to any one of Inventive Concepts 1-25, wherein testing the extraction liquid for the presence of the virus includes testing the extraction liquid for the presence of a virus that may be present in an upper respiratory tract.
Inventive Concept 39. The method according to Inventive Concept 38, wherein the virus that may be present in the upper respiratory tract is one or more viruses selected from the group of viruses consisting of: Epstein-Barr virus (EBV), Herpes simplex virus (HSV), a Human papillomavirus (HPV), an Influenza virus, a coronavirus, and a respiratory syncytial virus (RSV).
Inventive Concept 40. The method according to Inventive Concept 39, wherein the virus is the Influenza virus.
Inventive Concept 41. The method according to Inventive Concept 40, wherein the Influenza virus is one or more viruses selected from the group of viruses consisting of: Influenza A, Influenza B, Influenza C, and Influenza D.
Inventive Concept 42. The method according to Inventive Concept 39, wherein the virus is the coronavirus.
Inventive Concept 43. The method according to Inventive Concept 42, wherein the coronavirus is one or more viruses selected from the group of viruses consisting of: SARS-CoV-2, MERS-CoV, and SARS-CoV.
Inventive Concept 44. The method according to Inventive Concept 39, wherein the virus is the respiratory syncytial virus (RSV).
Inventive Concept 45. The method according to any one of Inventive Concepts 1-25, wherein testing the extraction liquid includes performing an immunoassay on the extraction liquid.
Inventive Concept 46. The method according to Inventive Concept 45, wherein performing the immunoassay includes performing a lateral flow immunoassay on the extraction liquid.
Inventive Concept 47. The method according to Inventive Concept 45, wherein performing the immunoassay includes performing an enzyme-linked immunosorbent assay (ELISA) on the extraction liquid.
Inventive Concept 48. The method according to any one of Inventive Concepts 1-25, wherein testing the extraction liquid includes performing a molecular-based assay on the extraction liquid.
Inventive Concept 49. The method according to Inventive Concept 48, wherein performing the molecular-based assay on the extraction liquid includes performing nucleic acid hybridization.
Inventive Concept 50. The method according to Inventive Concept 48, wherein performing the molecular-based assay on the extraction liquid includes performing nucleic acid amplification.
Inventive Concept 51. The method according to Inventive Concept 50, wherein the nucleic acid amplification is selected from the group consisting of: polymerase chain reaction (PCR) amplification, real-time quantitative PCR (qPCR) amplification, reverse transcriptase PCR (RT-PCR) amplification, and isothermal amplification.
Inventive Concept 52. The method according to any one of Inventive Concepts 1-25, wherein extracting the cellular components of the epithelial cells includes applying an extraction reagent to the epithelial cells.
Inventive Concept 53. The method according to Inventive Concept 52, wherein the extraction reagent includes one or more components selected from the group consisting of: one or more detergents, one or more salt solutions, one or more hypertonic solutions, one or more hypotonic solutions, one or more reducing agents, one or more chelating agents, one or more protease inhibitors, one or more proteases, one or more alkaline lysing agents, one or more enzymatic lysing agents, and one or more buffers.
Inventive Concept 54. The method according to Inventive Concept 53, wherein the extraction reagent includes one or more detergents selected from the group consisting of: an ionic detergent, a non-ionic detergent, and a chaotropic detergent.
Inventive Concept 55. The method according to Inventive Concept 54, wherein the extraction reagent includes one or more ionic detergents selected from the group consisting of: Sodium deoxycholate, sodium dodecyl sulphate (SDS), cetyltrimethylammonium bromide (CTAB), CHAPS, and CHAPSO.
Inventive Concept 56. The method according to Inventive Concept 54, wherein the extraction reagent includes one or more non-ionic detergents selected from the group consisting of: Triton-X-100, Triton-X-114, NP-40, polysorbate-20, polysorbate 80, and Octyl glucoside.
Inventive Concept 57. The method according to Inventive Concept 54, wherein the extraction reagent includes one or more chaotropic detergents selected from the group consisting of: urea, guanidine, and ethylenediaminetetraacetic acid (EDTA).
Inventive Concept 58. The method according to Inventive Concept 53, wherein the extraction reagent includes one or more hypertonic solutions selected from the group consisting of: Sodium chloride, Potassium chloride, and Sodium orthovanadate.
Inventive Concept 59. The method according to Inventive Concept 53, wherein the extraction reagent includes one or more hypotonic solutions selected from the group consisting of: Sodium chloride, Potassium chloride, Sodium orthovanadate, and pure water.
Inventive Concept 60. The method according to Inventive Concept 53, wherein the extraction reagent includes one or more reducing agents selected from the group consisting of: dithiothreitol (DTT), 2-Mercaptoethanol, Sodium dithionate, Sodium borohydride, Sodium thiosulfate, and Cysteine.
Inventive Concept 61. The method according to Inventive Concept 53, wherein the extraction reagent includes one or more chelating agents selected from the group consisting of: EDTA, EGTA, and DMPS.
Inventive Concept 62. The method according to Inventive Concept 53, wherein the extraction reagent includes one or more protease inhibitors selected from the group consisting of: PMSF, Aminocaproic acid, Pepstatin, Aprotinin, and Leupeptin.
Inventive Concept 63. The method according to Inventive Concept 53, wherein the extraction reagent includes one or more proteases selected from the group consisting of:

Proteinase K, Trypsin, Chymotrypsin, and Endoproteinase Glu-C (V8 protease).
Inventive Concept 64. The method according to Inventive Concept 53, wherein the extraction reagent includes one or more buffers selected from the group consisting of: Phosphate buffer, Tris buffer, HEPES, MES buffer, Borate buffer, Acetate buffer, and Carbonate buffer.
Inventive Concept 65. The method according to Inventive Concept 52, wherein applying the extraction reagent to the epithelial cells includes applying the extraction reagent to at least one of the one or more porous filters.
Inventive Concept 66. The method according to any one of Inventive Concepts 1-25, wherein extracting the cellular components of the epithelial cells includes applying sonication to the epithelial cells.
Inventive Concept 67. The method according to Inventive Concept 66, wherein applying the sonication to the epithelial cells includes applying the sonication to at least one of the one or more porous filters.
Inventive Concept 68. The method according to any one of Inventive Concepts 1-25, wherein extracting the cellular components of the epithelial cells includes applying an electrical field to the epithelial cells.
Inventive Concept 69. The method according to Inventive Concept 68, wherein applying the electrical field to the epithelial cells includes applying the electrical field to at least one of the one or more porous filters.
Inventive Concept 70. The method according to any one of Inventive Concepts 1-25, wherein extracting the cellular components of the epithelial cells includes changing a temperature of the epithelial cells.
Inventive Concept 71. The method according to Inventive Concept 70, wherein changing the temperature of the epithelial cells includes changing a temperature of at least one of the one or more porous filters.
Inventive Concept 72. The method according to Inventive Concept 70, wherein changing the temperature of the epithelial cells includes heating the epithelial cells.
Inventive Concept 73. The method according to Inventive Concept 72, wherein heating the epithelial cells includes heating at least one of the one or more porous filters.
Inventive Concept 74. The method according to Inventive Concept 70, wherein changing the temperature of the epithelial cells includes freezing and thawing the epithelial cells.
Inventive Concept 75. The method according to Inventive Concept 72, wherein freezing and thawing the epithelial cells includes freezing and thawing at least one of the one or more porous filters.
Inventive Concept 76. The method according to any one of Inventive Concepts 1-25, wherein extracting the cellular components of the epithelial cells includes physically extracting the cellular components of the epithelial cells.
Inventive Concept 77. The method according to Inventive Concept 76, wherein physically extracting the cellular components of the epithelial cells includes mechanically extracting the cellular components of the epithelial cells.
Inventive Concept 78. The method according to Inventive Concept 76, wherein physically extracting the cellular components of the epithelial cells includes manually extracting the cellular components of the epithelial cells.
Inventive Concept 79. The method according to Inventive Concept 76, wherein physically extracting the cellular components of the epithelial cells includes applying a physical extraction technique to at least one of the one or more porous filters.
Inventive Concept 80. The method according to Inventive Concept 79, wherein applying the physical extraction technique to the at least one of the one or more porous filters includes agitating the at least one of the one or more porous filters.
Inventive Concept 81. The method according to Inventive Concept 79, wherein applying the physical extraction technique to the at least one of the one or more porous filters includes vibrating the at least one of the one or more porous filters.
Inventive Concept 82. The method according to Inventive Concept 79, wherein applying the physical extraction technique to the at least one of the one or more porous filters includes macerating the at least one of the one or more porous filters.
Inventive Concept 83. The method according to Inventive Concept 82, wherein testing the extraction liquid includes testing the extraction liquid, including particles of the at least one of the one or more porous filters suspended or partially dissolved in the extraction liquid.
Inventive Concept 84. The method according to Inventive Concept 79, wherein applying the physical extraction technique to the at least one of the one or more porous filters includes tilting the at least one of the one or more porous filters.
Inventive Concept 85. The method according to any one of Inventive Concepts 1-25, wherein passing the non-centrifuged oronasopharyngeal fluid sample through the one or more porous filters includes passing the non-centrifuged oronasopharyngeal fluid sample in series through first and second porous filters having first and second average absolute pore sizes, respectively, the first average absolute pore size greater than the second absolute pore size.
Inventive Concept 86. The method according to Inventive Concept 85, wherein the first average absolute pore size is between 1.2 and 20 microns, and the second average absolute pore size is between 0.01 and 0.3 microns.
Inventive Concept 87. The method according to Inventive Concept 85, passing the non-centrifuged oronasopharyngeal fluid sample in series through the first and the second porous filters includes:

passing the non-centrifuged oronasopharyngeal fluid sample through the first porous filter to separate the epithelial cells from the non-centrifuged oronasopharyngeal fluid sample by size-based filtration, and to allow a filtrate to pass through the first porous filter, the filtrate including free viral particles; and

passing the filtrate through the second porous filter to separate the free viral particles from the filtrate by size-based filtration.
Inventive Concept 88. The method according to Inventive Concept 87, wherein testing the extraction liquid includes testing the extraction liquid while the extraction liquid is in contact with at least the second porous filter.
Inventive Concept 89. The method according to Inventive Concept 88, wherein testing the extraction liquid includes testing the extraction liquid while the extraction liquid is in contact with at least the first and the second porous filters.
Inventive Concept 90. The method according to Inventive Concept 88, wherein testing the extraction liquid while the extraction liquid is in contact with at least the first and the second porous filters includes testing a first extraction liquid while the first extraction liquid is in contact with at least the first porous filter and testing a second extraction liquid while the second extraction liquid is in contact with at least the second porous filter.
Inventive Concept 91. The method according to any one of Inventive Concepts 1-25,
   wherein the one or more porous filters are one or more cell-trapping filters, and
   wherein testing the extraction liquid for the presence of the virus includes:
      passing the extraction liquid through a virus-trapping filter to trap at least a portion of free viral particles; and
      thereafter, testing for the presence of the free viral particles trapped by the virus-trapping filter.
Inventive Concept 92. The method according to Inventive Concept 91, wherein the virus-trapping filter includes a mechanical filter, and wherein passing the extraction liquid through the virus-trapping filter includes passing the extraction liquid through the virus mechanical filter to trap the at least a portion of the free viral particles by size-based filtration.
Inventive Concept 93. The method according to Inventive Concept 91, wherein the virus-trapping filter includes fixed antibodies configured to capture the free viral particles by affinity-based filtration.
Inventive Concept 94. The method according to any one of Inventive Concepts 1-25,

wherein passing the non-centrifuged oronasopharyngeal fluid sample through the one or more porous filters includes passing the non-centrifuged oronasopharyngeal fluid sample through the one or more porous filters while the one or more porous filters are within a testing device, and

wherein extracting the cellular components of the epithelial cells includes extracting the cellular components of the epithelial cells while the epithelial cells and the one or more porous filters are within the testing device.
Inventive Concept 95. The method according to Inventive Concept 94, wherein extracting the cellular components of the epithelial cells includes applying at least one extraction agent to the one or more porous filters while the one or more porous filters are within the testing device.
Inventive Concept 96. The method according to Inventive Concept 94, wherein testing the extraction liquid includes testing the extraction liquid while the extraction liquid is within the testing device.
Inventive Concept 97. The method according to Inventive Concept 96, wherein testing the extraction liquid for the presence of the virus includes inserting a test strip into the testing device.
Inventive Concept 98. The method according to Inventive Concept 94, wherein testing the extraction liquid includes removing the extraction liquid from the testing device and thereafter testing the extraction liquid.
Inventive Concept 99. The method according to Inventive Concept 98, wherein testing the extraction liquid includes removing the extraction liquid and at least a portion of the filter from the testing device and thereafter testing the extraction liquid and the at least a portion of the filter.
Inventive Concept 100. The method according to any one of Inventive Concepts 1-25,

wherein passing the non-centrifuged oronasopharyngeal fluid sample through the one or more porous filters includes passing the non-centrifuged oronasopharyngeal fluid sample through the one or more porous filters while the one or more porous filters are within a testing device, and

wherein extracting the cellular components of the epithelial cells includes removing at least a portion of the one or more porous filters from the testing device and thereafter extracting the cellular components of the epithelial cells while the epithelial cells and the at least a portion of the one or more porous filters are outside the testing device.
Inventive Concept 101. The method according to Inventive Concept 100, wherein testing the extraction liquid for the presence of the virus includes performing a detection test on the extraction liquid outside the testing device.
Inventive Concept 102. The method according to Inventive Concept 101, wherein testing the extraction liquid for the presence of the virus includes performing a nucleic acid amplification detection test on the extraction liquid outside the testing device.
Inventive Concept 103. The method according to any one of Inventive Concepts 1-25, wherein testing the extraction liquid for the presence of the virus includes completing ascertaining whether the virus is present within five hours of beginning passing the non-centrifuged oronasopharyngeal fluid sample through the one or more porous filters.
Inventive Concept 104. The method according to Inventive Concept 103, wherein completing ascertaining whether the virus is present includes completing ascertaining whether the virus is present within two hours of beginning passing the non-centrifuged oronasopharyngeal fluid sample through the one or more porous filters.
Inventive Concept 105. The method according to Inventive Concept 103, wherein completing ascertaining whether the virus is present includes completing ascertaining whether the virus is present at least 5 minutes after and within 20 minutes of beginning passing the non-centrifuged oronasopharyngeal fluid sample through the one or more porous filters.
Inventive Concept 106. The method according to Inventive Concept 105, wherein completing ascertaining whether the virus is present includes completing ascertaining whether the virus is present at least 8 minutes after and within 20 minutes of beginning passing the non-centrifuged oronasopharyngeal fluid sample through the one or more porous filters.
Inventive Concept 107. The method according to Inventive Concept 106, wherein completing ascertaining whether the virus is present includes completing ascertaining whether the virus is present at least 12 minutes after and within 20 minutes of beginning passing the non-centrifuged oronasopharyngeal fluid sample through the one or more porous filters.
Inventive Concept 108. The method according to Inventive Concept 107, wherein completing ascertaining whether the virus is present includes completing ascertaining whether the virus is present at least 15 minutes after and within 20 minutes of beginning passing the non-centrifuged oronasopharyngeal fluid sample through the one or more porous filters.
Inventive Concept 109. The method according to any one of Inventive Concepts 1-25, further including, before passing the non-centrifuged oronasopharyngeal fluid sample through the one or more porous filters, collecting the fluid sample from the human or non-human animal.

There is further provided, in accordance with an Inventive Concept 110 of the present invention, a method for testing a non-centrifuged oronasopharyngeal fluid sample taken from a human or non-human animal for the presence of a cellular biomarker, the method including:

passing the non-centrifuged oronasopharyngeal fluid sample through one or more porous filters to separate animal cells from the non-centrifuged oronasopharyngeal fluid sample by size-based filtration;

thereafter, preparing an extraction liquid by extracting cellular components of the animal cells separated from the non-centrifuged oronasopharyngeal fluid sample; and

thereafter, testing the extraction liquid for the presence of the cellular biomarker.
Inventive Concept 111. The method according to Inventive Concept 110, wherein testing the extraction liquid includes testing the extraction liquid while the extraction liquid is in contact with at least one of the one or more porous filters.
Inventive Concept 112. The method according to Inventive Concept 110, wherein testing the extraction liquid for the presence of the cellular biomarker includes removing at least a portion of the extraction liquid from contact with the one or more porous filters, and testing at least a portion of the removed extraction liquid.
Inventive Concept 113. The method according to Inventive Concept 110, wherein testing the extraction liquid for the presence of the cellular biomarker includes applying a transport liquid to at least one of the one or more porous filters to produce a mixture of the extraction liquid and the transport liquid, removing a portion of the mixture from the at least one of the one or more porous filters, and testing the removed mixture for the presence of the cellular biomarker.
Inventive Concept 114. The method according to Inventive Concept 110, wherein preparing the extraction liquid includes extracting, while the animal cells are in contact with at least one of the one or more porous filters, the cellular components of the animal cells separated from the non-centrifuged oronasopharyngeal fluid sample.
Inventive Concept 115. The method according to Inventive Concept 110, wherein testing for presence of the cellular biomarker includes quantifying a level of the cellular biomarker.
Inventive Concept 116. The method according to Inventive Concept 110, wherein filtering the non-centrifuged oronasopharyngeal fluid sample includes filtering between 0.5 and 50 cc of the non-centrifuged oronasopharyngeal fluid sample.
Inventive Concept 117. The method according to Inventive Concept 116, wherein filtering the non-centrifuged oronasopharyngeal fluid sample includes filtering between 4 and 25 cc of the non-centrifuged oronasopharyngeal fluid sample.
Inventive Concept 118. The method according to Inventive Concept 110, wherein the non-centrifuged oronasopharyngeal fluid sample is also non-cultured, and wherein passing the non-centrifuged oronasopharyngeal fluid sample through the one or more porous filters includes passing the non-centrifuged and non-cultured oronasopharyngeal fluid sample through the one or more porous filters.
Inventive Concept 119. The method according to Inventive Concept 110, wherein at least one of the one or more porous filters has an average absolute pore size of between 0.01 and 20 microns.
Inventive Concept 120. The method according to Inventive Concept 119, wherein the average absolute pore size is between 0.1 and 20 microns.
Inventive Concept 121. The method according to Inventive Concept 119, wherein the average absolute pore size is between 0.45 and 9.0 microns.
Inventive Concept 122. The method according to Inventive Concept 119, wherein the average absolute pore size is between 1.2 and 20 microns.
Inventive Concept 123. The method according to Inventive Concept 122, wherein the average absolute pore size is between 2.0 and 20 microns.
Inventive Concept 124. The method according to Inventive Concept 123, wherein the average absolute pore size is between 5.0 and 20 microns.
Inventive Concept 125. The method according to Inventive Concept 121, wherein the average absolute pore size is between 0.45 and 2.0 microns.
Inventive Concept 126. The method according to Inventive Concept 110, wherein at least one of the one or more porous filters has an average nominal pore size of between 0.01 and 20 microns.
Inventive Concept 127. The method according to Inventive Concept 126, wherein the average nominal pore size is between 0.1 and 20 microns.
Inventive Concept 128. The method according to Inventive Concept 127, wherein the average nominal pore size is between 0.45 and 9.0 microns.
Inventive Concept 129. The method according to Inventive Concept 126, wherein the average nominal pore size is between 1.2 and 20 microns.
Inventive Concept 130. The method according to Inventive Concept 129, wherein the average nominal pore size is between 1.2 and 20 microns.
Inventive Concept 131. The method according to Inventive Concept 130, wherein the average nominal pore size is between 5.0 and 20 microns.
Inventive Concept 132. The method according to Inventive Concept 128, wherein the average nominal pore size is between 0.45 and 2.0 microns.
Inventive Concept 133. The method according to Inventive Concept 110, wherein extracting the cellular components of the animal cells includes lysing the animal cells.
Inventive Concept 134. The method according to Inventive Concept 110, further including, after passing the non-centrifuged oronasopharyngeal fluid sample through the one or more porous filters and before preparing the extraction liquid, adding, to at least one of the one or more porous filters, material from a nasal swab or a nasopharyngeal swab of the human or non-human animal.
Inventive Concept 135. The method according to any one of Inventive Concepts 110-134, wherein the animal cells include epithelial cells, and wherein passing the non-centrifuged oronasopharyngeal fluid sample through the one or more porous filters includes passing the non-centrifuged oronasopharyngeal fluid sample through the one or more porous filters to separate the epithelial cells from the non-centrifuged oronasopharyngeal fluid sample by size-based filtration.
Inventive Concept 136. The method according to any one of Inventive Concepts 110-134, wherein the animal cells include leukocytes, and wherein passing the non-centrifuged oronasopharyngeal fluid sample through the one or more porous filters includes passing the non-centrifuged oronasopharyngeal fluid sample through the one or more porous filters to separate the leukocytes from the non-centrifuged oronasopharyngeal fluid sample by size-based filtration.
Inventive Concept 137. The method according to any one of Inventive Concepts 110-134,

wherein preparing the extraction liquid includes extracting and exposing a cellular biomarker, and

wherein testing the extraction liquid for the presence of the cellular biomarker includes testing the extraction liquid for the presence of the cellular biomarker.
Inventive Concept 138. The method according to Inventive Concept 137,

wherein extracting and exposing the cellular biomarker includes extracting and exposing a tumor-specific cellular biomarker, and

wherein testing the extraction liquid for the presence of the cellular biomarker includes testing the extraction liquid for the presence of the tumor-specific cellular biomarker.
Inventive Concept 139. The method according to Inventive Concept 137,

wherein extracting and exposing the cellular biomarker includes extracting and exposing an inflammatory-specific cellular biomarker, and

wherein testing the extraction liquid for the presence of the cellular biomarker includes testing the extraction liquid for the presence of the inflammatory-specific cellular biomarker.
Inventive Concept 140. The method according to Inventive Concept 137,

wherein extracting and exposing the cellular biomarker includes extracting and exposing a disease-specific cellular biomarker, and

wherein testing the extraction liquid for the presence of the cellular biomarker includes testing the extraction liquid for the presence of the disease-specific cellular biomarker.
Inventive Concept 141. The method according to any one of Inventive Concepts 110-134, wherein the non-centrifuged oronasopharyngeal fluid sample taken from the human or non-human animal is one or more non-centrifuged oronasopharyngeal fluid samples taken from one or more of oronasopharyngeal cavities of the human or non-human animal.
Inventive Concept 142. The method according to Inventive Concept 141, wherein the non-centrifuged oronasopharyngeal fluid sample includes a non-centrifuged nasal wash taken from the human or non-human animal.
Inventive Concept 143. The method according to Inventive Concept 141, wherein the non-centrifuged oronasopharyngeal fluid sample includes non-centrifuged nasal aspirate taken from the human or non-human animal.
Inventive Concept 144. The method according to Inventive Concept 141, wherein the non-centrifuged oronasopharyngeal fluid sample includes non-centrifuged gargled fluid taken from the human or non-human animal.
Inventive Concept 145. The method according to Inventive Concept 141, wherein the non-centrifuged oronasopharyngeal fluid sample includes a non-centrifuged oral wash taken from the human or non-human animal.
Inventive Concept 146. The method according to Inventive Concept 141, wherein the non-centrifuged oronasopharyngeal fluid sample includes saliva taken from the human or non-human animal.
Inventive Concept 147. The method according to Inventive Concept 141, wherein the non-centrifuged oronasopharyngeal fluid sample includes (a) material from (i) a nasal swab or (ii) a nasopharyngeal swab mixed with (b) (i) non-centrifuged gargled fluid taken from the human or non-human animal or (ii) nasal wash taken from the human or non-human animal.
Inventive Concept 148. The method according to any one of Inventive Concepts 110-134, wherein testing the extraction liquid includes performing an immunoassay on the extraction liquid.
Inventive Concept 149. The method according to Inventive Concept 148, wherein performing the immunoassay includes performing a lateral flow immunoassay on the extraction liquid.
Inventive Concept 150. The method according to Inventive Concept 148, wherein performing the immunoassay includes performing an enzyme-linked immunosorbent assay (ELISA) on the extraction liquid.
Inventive Concept 151. The method according to any one of Inventive Concepts 110-134, wherein testing the extraction liquid includes performing a molecular-based assay on the extraction liquid.
Inventive Concept 152. The method according to Inventive Concept 151, wherein performing the molecular-based assay on the extraction liquid includes performing nucleic acid hybridization.
Inventive Concept 153. The method according to Inventive Concept 151, wherein performing the molecular-based assay on the extraction liquid includes performing nucleic acid amplification.
Inventive Concept 154. The method according to Inventive Concept 153, wherein the nucleic acid amplification is selected from the group consisting of: polymerase chain reaction (PCR) amplification, real-time quantitative PCR (qPCR) amplification, reverse transcriptase PCR (RT-PCR) amplification, and isothermal amplification.
Inventive Concept 155. The method according to any one of Inventive Concepts 110-134, wherein testing the extraction liquid does not include performing nucleic acid amplification.
Inventive Concept 156. The method according to any one of Inventive Concepts 110-134, wherein extracting the cellular components of the animal cells includes applying an extraction reagent to the animal cells.
Inventive Concept 157. The method according to Inventive Concept 156, wherein the extraction reagent includes one or more components selected from the group consisting of:

one or more detergents, one or more hypertonic solutions, one or more hypotonic solutions, one or more reducing agents, one or more chelating agents, one or more protease inhibitors, one or more proteases, and one or more buffers.
Inventive Concept 158. The method according to Inventive Concept 157, wherein the extraction reagent includes one or more detergents selected from the group consisting of: an ionic detergent, a non-ionic detergent, and a chaotropic detergent.
Inventive Concept 159. The method according to Inventive Concept 158, wherein the extraction reagent includes one or more ionic detergents selected from the group consisting of: Sodium deoxycholate, sodium dodecyl sulphate (SDS), cetyltrimethylammonium bromide (CTAB), CHAPS, and CHAPSO.
Inventive Concept 160. The method according to Inventive Concept 158, wherein the extraction reagent includes one or more non-ionic detergents selected from the group consisting of: Triton-X-100, Triton-X-114, NP-40, polysorbate-20, polysorbate 80, and Octyl glucoside.
Inventive Concept 161. The method according to Inventive Concept 158, wherein the extraction reagent includes one or more chaotropic detergents selected from the group consisting of: urea, guanidine, and ethylenediaminetetraacetic acid (EDTA).
Inventive Concept 162. The method according to Inventive Concept 157, wherein the extraction reagent includes one or more hypertonic solutions selected from the group consisting of: Sodium chloride, Potassium chloride, and Sodium orthovanadate.
Inventive Concept 163. The method according to Inventive Concept 157, wherein the extraction reagent includes one or more hypotonic solutions selected from the group consisting of: Sodium chloride, Potassium chloride, Sodium orthovanadate, and pure water.
Inventive Concept 164. The method according to Inventive Concept 157, wherein the extraction reagent includes one or more reducing agents selected from the group consisting of: dithiothreitol (DTT), 2-Mercaptoethanol, Sodium dithionate, Sodium borohydride, Sodium thiosulfate, and Cysteine.
Inventive Concept 165. The method according to Inventive Concept 157, wherein the extraction reagent includes one or more chelating agents selected from the group consisting of: EDTA, EGTA, and DMPS.
Inventive Concept 166. The method according to Inventive Concept 157, wherein the extraction reagent includes one or more protease inhibitors selected from the group consisting of: PMSF, Aminocaproic acid, Pepstatin, Aprotinin, and Leupeptin.
Inventive Concept 167. The method according to Inventive Concept 157, wherein the extraction reagent includes one or more proteases selected from the group consisting of: Proteinase K, Trypsin, Chymotrypsin, and Endoproteinase Glu-C (V8 protease).
Inventive Concept 168. The method according to Inventive Concept 157, wherein the extraction reagent includes one or more buffers selected from the group consisting of: Phosphate buffer, Tris buffer, HEPES, MES buffer, Borate buffer, Acetate buffer, and Carbonate buffer.
Inventive Concept 169. The method according to Inventive Concept 156, wherein applying the extraction reagent to the animal cells includes applying the extraction reagent to at least one of the one or more porous filters.
Inventive Concept 170. The method according to any one of Inventive Concepts 110-134, wherein extracting the cellular components of the animal cells includes applying sonication to the animal cells.
Inventive Concept 171. The method according to Inventive Concept 170, wherein applying the sonication to the animal cells includes applying the sonication to at least one of the one or more porous filters.
Inventive Concept 172. The method according to any one of Inventive Concepts 110-134, wherein extracting the cellular components of the animal cells includes applying an electrical field to the animal cells.
Inventive Concept 173. The method according to Inventive Concept 172, wherein applying the electrical field to the animal cells includes applying the electrical field to at least one of the one or more porous filters.
Inventive Concept 174. The method according to any one of Inventive Concepts 110-134, wherein extracting the cellular components of the animal cells includes changing a temperature of the animal cells.
Inventive Concept 175. The method according to Inventive Concept 174, wherein changing the temperature of the animal cells includes changing a temperature of at least one of the one or more porous filters.
Inventive Concept 176. The method according to Inventive Concept 174, wherein changing the temperature of the animal cells includes heating the animal cells.
Inventive Concept 177. The method according to Inventive Concept 176, wherein heating the animal cells includes heating at least one of the one or more porous filters.
Inventive Concept 178. The method according to Inventive Concept 174, wherein changing the temperature of the animal cells includes freezing and thawing the animal cells.
Inventive Concept 179. The method according to Inventive Concept 176, wherein freezing and thawing the animal cells includes freezing and thawing at least one of the one or more porous filters.
Inventive Concept 180. The method according to any one of Inventive Concepts 110-134, wherein extracting the cellular components of the animal cells includes physically extracting the cellular components of the animal cells.
Inventive Concept 181. The method according to Inventive Concept 180, wherein physically extracting the cellular components of the animal cells includes mechanically extracting the cellular components of the animal cells.
Inventive Concept 182. The method according to Inventive Concept 180, wherein physically extracting the cellular components of the animal cells includes manually extracting the cellular components of the animal cells.
Inventive Concept 183. The method according to Inventive Concept 180, wherein physically extracting the cellular components of the animal cells includes applying a physical extraction technique to at least one of the one or more porous filters.
Inventive Concept 184. The method according to Inventive Concept 183, wherein applying the physical extraction technique to the at least one of the one or more porous filters includes agitating the at least one of the one or more porous filters.
Inventive Concept 185. The method according to Inventive Concept 183, wherein applying the physical extraction technique to the at least one of the one or more porous filters includes vibrating the at least one of the one or more porous filters.
Inventive Concept 186. The method according to Inventive Concept 183, wherein applying the physical extraction technique to the at least one of the one or more porous filters includes macerating the at least one of the one or more porous filters.
Inventive Concept 187. The method according to Inventive Concept 186, wherein testing the extraction liquid includes testing the extraction liquid, including particles of the at least one of the one or more porous filters suspended or partially dissolved in the extraction liquid.
Inventive Concept 188. The method according to Inventive Concept 183, wherein applying the physical extraction technique to the at least one of the one or more porous filters includes tilting the at least one of the one or more porous filters.
Inventive Concept 189. The method according to any one of Inventive Concepts 110-134,

wherein passing the non-centrifuged oronasopharyngeal fluid sample through the one or more porous filters includes passing the non-centrifuged oronasopharyngeal fluid sample through the one or more porous filters while the one or more porous filters are within a testing device, and

wherein extracting the cellular components of the animal cells includes extracting the cellular components of the animal cells while the animal cells and the one or more porous filters are within the testing device.
Inventive Concept 190. The method according to Inventive Concept 189, wherein extracting the cellular components of the animal cells includes applying at least one extraction agent to the one or more porous filters while the one or more porous filters are within the testing device.
Inventive Concept 191. The method according to Inventive Concept 189, wherein testing the extraction liquid includes testing the extraction liquid while the extraction liquid is within the testing device.
Inventive Concept 192. The method according to Inventive Concept 191, wherein testing the extraction liquid for the presence of the cellular biomarker includes inserting a test strip into the testing device.
Inventive Concept 193. The method according to Inventive Concept 189, wherein testing the extraction liquid includes removing the extraction liquid from the testing device and thereafter testing the extraction liquid.
Inventive Concept 194. The method according to Inventive Concept 193, wherein testing the extraction liquid includes removing the extraction liquid and at least a portion of the filter from the testing device and thereafter testing the extraction liquid and the at least a portion of the filter.
Inventive Concept 195. The method according to any one of Inventive Concepts 110-134,

wherein passing the non-centrifuged oronasopharyngeal fluid sample through the one or more porous filters includes passing the non-centrifuged oronasopharyngeal fluid sample through the one or more porous filters while the one or more porous filters are within a testing device, and

wherein extracting the cellular components of the animal cells includes removing at least a portion of the one or more porous filters from the testing device and thereafter extracting the cellular components of the animal cells while the animal cells and the at least a portion of the one or more porous filters are outside the testing device.
Inventive Concept 196. The method according to Inventive Concept 195, wherein testing the extraction liquid for the presence of the cellular biomarker includes performing a detection test on the extraction liquid outside the testing device.
Inventive Concept 197. The method according to Inventive Concept 196, wherein testing the extraction liquid for the presence of the cellular biomarker includes performing a nucleic acid amplification detection test on the extraction liquid outside the testing device.
Inventive Concept 198. The method according to any one of Inventive Concepts 110-134, wherein testing the extraction liquid for the presence of the cellular biomarker includes completing ascertaining whether the cellular biomarker is present within five hours of beginning passing the non-centrifuged oronasopharyngeal fluid sample through the one or more porous filters.
Inventive Concept 199. The method according to Inventive Concept 198, wherein completing ascertaining whether the cellular biomarker is present includes completing ascertaining whether the cellular biomarker is present within two hours of beginning passing the non-centrifuged oronasopharyngeal fluid sample through the one or more porous filters.
Inventive Concept 200. The method according to Inventive Concept 198, wherein completing ascertaining whether the cellular biomarker is present includes completing ascertaining whether the cellular biomarker is present at least 5 minutes after and within 20 minutes of beginning passing the non-centrifuged oronasopharyngeal fluid sample through the one or more porous filters.
Inventive Concept 201. The method according to Inventive Concept 200, wherein completing ascertaining whether the cellular biomarker is present includes completing ascertaining whether the cellular biomarker is present at least 8 minutes after and within 20 minutes of beginning passing the non-centrifuged oronasopharyngeal fluid sample through the one or more porous filters.
Inventive Concept 202. The method according to Inventive Concept 201, wherein completing ascertaining whether the cellular biomarker is present includes completing ascertaining whether the cellular biomarker is present at least 12 minutes after and within 20 minutes of beginning passing the non-centrifuged oronasopharyngeal fluid sample through the one or more porous filters.
Inventive Concept 203. The method according to Inventive Concept 202, wherein completing ascertaining whether the cellular biomarker is present includes completing ascertaining whether the cellular biomarker is present at least 15 minutes after and within 20 minutes of beginning passing the non-centrifuged oronasopharyngeal fluid sample through the one or more porous filters.
Inventive Concept 204. The method according to any one of Inventive Concepts 110-134, further including, before passing the non-centrifuged oronasopharyngeal fluid sample through the one or more porous filters, collecting the fluid sample from the human or non-human animal.
Inventive Concept 205. The method according to any one of Inventive Concepts 110-134, wherein the cellular biomarker does not include nucleic acid, and wherein testing the extraction liquid for the presence of the cellular biomarker includes testing the extraction liquid for the presence of the cellular biomarker that does not include nucleic acid.

There is still further provided, in accordance with an Inventive Concept 206 of the present invention, a method for testing a non-centrifuged oronasopharyngeal fluid sample taken from a human or non-human animal for the presence of at least cellular components of animal cells, bacteria, and free viral particles, the method including:

passing the non-centrifuged oronasopharyngeal fluid sample through a cell-trapping porous filter to separate the animal cells from the non-centrifuged oronasopharyngeal fluid sample by size-based filtration and to allow a first filtrate to pass through the cell-trapping porous filter, the first filtrate potentially including the bacteria and the free viral particles;

passing the first filtrate through a bacteria-trapping porous filter to separate the bacteria from the non-centrifuged oronasopharyngeal fluid sample by size-based filtration and to allow a second filtrate to pass through the bacteria porous filter, the second filtrate potentially including the free viral particles;

passing the second filtrate through a virus-trapping porous filter to separate the free viral particles from the non-centrifuged oronasopharyngeal fluid sample;

thereafter, preparing at least:
(a) a first extraction liquid by extracting the cellular components of the animal cells separated from the non-centrifuged oronasopharyngeal fluid sample by the cell-trapping porous filter,
(b) a second extraction liquid by extracting a bacterial target from the bacteria separated from first filtrate by the bacteria-trapping porous filter, and
(c) a third extraction liquid by extracting a viral target from the free viral particles separated from the second filtrate by the virus-trapping porous filter; and thereafter:
(a) testing the first extraction liquid,
(b) testing the second extraction liquid for the presence of the bacteria, and
(c) testing the third extraction liquid for the presence of the free viral particles.
Inventive Concept 207. The method according to Inventive Concept 206, wherein testing the first extraction liquid includes testing the first extraction liquid for the presence of a virus.
Inventive Concept 208. The method according to Inventive Concept 206, wherein testing the first extraction liquid includes testing the first extraction liquid for the presence of a cellular biomarker.

The present invention will be more fully understood from the following detailed description of embodiments thereof, taken together with the drawings, in which:

### BRIEF DESCRIPTION OF THE DRAWINGS

FIGS. 1A-H are schematic illustrations of a testing device for testing for presence of a particulate in a liquid, in accordance with an application of the present invention;

FIG. 2 is a schematic illustration of another testing device for testing for presence of a particulate in a liquid, in accordance with an application of the present invention;

FIGS. 3A-C are schematic illustrations of yet another testing device for testing for presence of a particulate in a liquid, in accordance with an application of the present invention;

FIGS. 4A-H are schematic illustrations of a testing device for testing for presence of a particulate in a liquid, in accordance with an application of the present invention;

FIGS. 5A-B are schematic illustrations of a testing device for testing for presence of a particulate in a liquid, in accordance with an application of the present invention;

FIGS. 6A-B are schematic illustrations of a testing device for testing for presence of a particulate in a liquid, in accordance with an application of the present invention;

FIGS. 7A-B are schematic illustrations of a testing device for testing for presence of a particulate in a liquid, in accordance with an application of the present invention;

FIGS. 8A-C are schematic illustrations of the testing device of FIGS. 1A-H further comprising one or more heating elements, in accordance with respective applications of the present invention;

FIG. 9 is a schematic illustration of a method for performing a test, in accordance with an application of the present invention;

FIGS. 10A-B are schematic illustrations of a device and method for testing for presence of a particulate in a liquid, in accordance with an application of the present invention;

FIG. 11 is a flow chart schematically depicting a method for testing a non-centrifuged oronasopharyngeal fluid sample taken from a body of a patient for the presence of a particulate, such as a virus or a cellular biomarker, in accordance with some applications of the present invention; and

FIGS. 12A-B are schematic and cross-sectional views of a filter chamber, respectively, in accordance with an application of the present invention.

### DETAILED DESCRIPTION OF APPLICATIONS

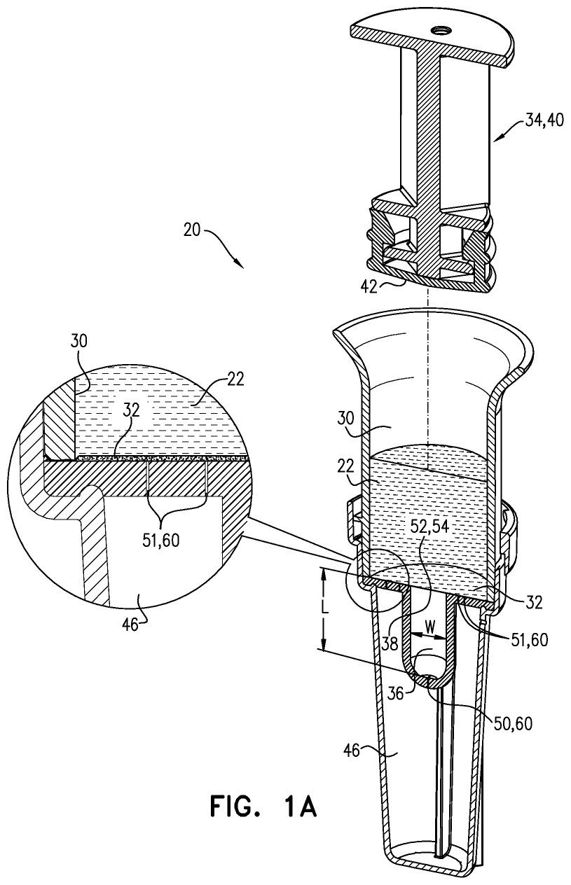

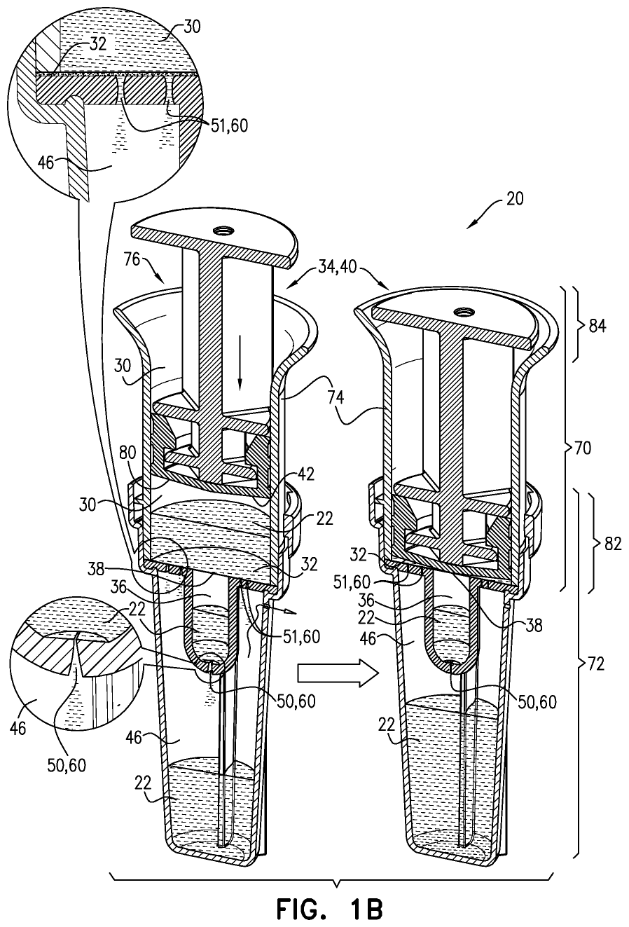

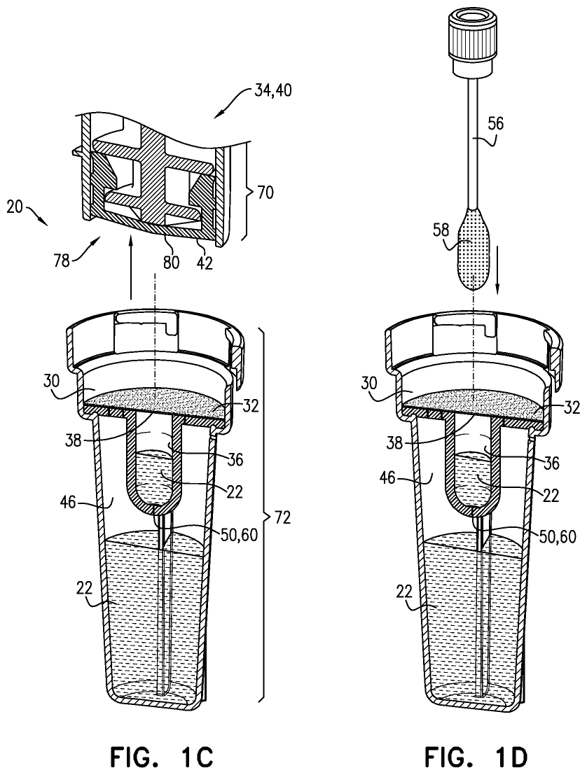

FIGS. 1A-H are schematic illustrations of a testing device 20 for testing for presence of particulate, such as a virus or a cellular biomarker, in a liquid 22, such as oronasopharyngeal fluid, in accordance with an application of the present invention. For some applications, the particulate comprises a biological particulate, for example, a virus (e.g., a free virus and/or a viral particle within a biological cell), a cellular biomarker (e.g., a tumor-specific cellular biomarker, an inflammatory-specific cellular biomarker, and/or a disease-specific cellular biomarker), a biological cell (e.g., cells containing a virus and/or a cellular biomarker, such as epithelial cells or leukocytes), a biological antigen (e.g., a protein antigen or a carbohydrate antigen that is a viral target or a cellular biomarker), and/or a nucleic acid (e.g., DNA or RNA, such as a viral DNA target or a viral RNA target).

Testing device 20 typically comprises:
a liquid container 30 for containing liquid 22; typically, liquid container 30 has an internal volume of at least 0.5 ml (e.g., at least 1 ml, such as at least 4 ml, such as at least 5 ml), no more than 500 ml (e.g., no more than 70 ml), and/or between 0.5 ml (e.g., 1 ml, 4 ml, or 5 ml) and 500 ml (e.g., 70 ml);
a filter 32, disposed in or downstream of liquid container 30; and
a liquid-pressure source 34, which is arranged to apply pressure to drive liquid 22 contained in liquid container 30 through filter 32.

As used in the present application, including in the claims and Inventive Concepts, “upstream” and “downstream” refer to the direction of fluid flow through testing device 20, and not the orientation of the device with respect to the Earth.

Filter 32 comprises synthetic or natural materials formed, for example, as a matrix, membrane, fabric, beads, or other configuration. For example, the inventors have tested the following filters manufactured by Sterlitech (Washington, USA):
Grade C glass microfiber filter media (Cat. No. C2500 & C3700)
GC-50 glass fiber membrane filters (Cat. No. GC5037100)
polyethersulfone (PES) membrane filters (Cat. No. PES0825100, PES0837100, PES1225100, PES1237100, PES06525100, PES4525100, PES4525100)
polycarbonate membrane filters (Cat. No. PCT0613100, PCT2025100, PCT0625100, PCT1025100, PCT0825100)
cellulose acetate membrane filters (Cat. No. CA0825100)
polyester membrane filters (Cat. No. PET0125100, PET0825100)

Typically, filter 32 is configured to trap at least 40% (such as at least 95%, e.g., at least 99%) of the particulate to be tested and allow passage of liquid 22. For example, for applications in which the particulate is epithelial cells that contain a virus, the filter may be configured to trap at least 40% (such as at least 95%, e.g., at least 99%) of the epithelial cells that contain a virus and allow passage of liquid 22. For example, for applications in which the particulate is cells (such as epithelial cells or leukocytes) that contain a cellular biomarker, the filter may be configured to trap at least 40% (such as at least 95%, e.g., at least 99%) of the cells that contain the cellular biomarker and allow passage of liquid 22. For some applications, filter 32 has a filter surface area of an upstream side of the filter equal to at least 0.3 cm2 (e.g., at least 1.7 cm2, such as at least 2.5 cm2), no more than 100 cm2 (e.g., no more than 30 cm2, such as no more than 20 cm2), and/or between 0.3 cm2 and 100 cm2, such as between 0.3 and 30 cm2, such as between 1.7 and 20 cm2, e.g., between 2.5 and 20 cm2. Since oronasopharyngeal fluid samples, such as gargled fluid and saliva not swabbed from the throat of a patient, tend have high viscosity and low flow rate, direct size-based filtration using oronasopharyngeal fluid with a volume of greater than 4 mL, a most-upstream filter having a surface area that is less than 1.7 cm2, and/or a most-upstream filter having pore size that is less than 1 micron often causes high filtration pressure and may cause the filter to become clogged during filtration. Therefore, in some applications, filter 32 has a filter surface area of at least 2.5 cm2, such as between 2.5 and 20 cm2, and an absolute or nominal pore size of at least 1 micron, such as between 1 and 20 microns. For some applications, a most-upstream filter of the one or more filters has a filter surface area of at least 2.5 cm2 and an absolute or nominal pore size of at least 1 micron.

For some applications, the nominal pore size is representative of a minimum size of spherical particles (e.g., inert spherical particles) necessary for the respective filter to retain 85% of the spherical particles when distilled H2O containing the spherical particles is passed through the respective filter at 20 degrees C. under pressure supplied by a 10 cm water column (of course, without downstream resistance) (herein, the “challenge-test nominal pore size”). It will be appreciated that the spherical particles, distilled H2O, and 10 cm water column are not elements of the device, but are instead non-device elements that are used to characterize certain physical properties of the filter(s), as is conventional for filter characterization in the filter art.

Alternatively, the nominal pore size may be characterized by a bubble point test, as is well known in the filter art, using a capillary flow porometer, such as CFP-1500A Automatic Capillary Flow Porometer (optionally, including options E, X, L, and/or M, if necessary for characterizing certain larger pore sizes) made by Porous Materials Inc. (PMI) (Ithaca, N.Y., USA), and a wetting liquid having a fluid surface tension of 15.9 dynes/cm, such as Galwick (PMI) (herein, the “bubble-point nominal pore size”).

As used in the present application, including in the claims and Inventive Concepts, a “pore” means any opening through a filter through which at least distilled H2O can pass.

For some applications, liquid-pressure source 34 comprises at least one of the following:
a plunger 40, which comprises a plunger head 42 that is shaped so as to be insertable into liquid container 30 so as to form a movable seal with a wall of a plunger housing (optionally, all or a portion of liquid container 30 defines the wall of the plunger housing);
a positive-pressure pump (e.g., a hydraulic pump, a syringe, or a motorized and/or electrical pump) disposed upstream of filter 32 (configuration not shown); optionally, for some application, the positive-pressure pump comprises a chamber with one or more flexible walls, the squeezing of which pumps air and/or liquid 22 itself out of the chamber; or
a vacuum pump disposed downstream of filter 32 (and, if provided, of the one or more valves 60, described hereinbelow) (configuration not shown).

For some applications, plunger 40 further comprises a plunger shaft, and plunger head 42 is disposed at a downstream end portion of the plunger shaft.

For some applications, testing device 20 further comprises a waste liquid receptacle 46, which is coupled to liquid container 30 downstream of filter 32 (and, if provided, of the one or more valves 60, described hereinbelow). Liquid-pressure source 34 is arranged to apply pressure to drive liquid 22 contained in liquid container 30 through filter 32 and then into waste liquid receptacle 46.

For some applications in which liquid 22 is a non-centrifuged oronasopharyngeal fluid sample, liquid 22 includes at least one substance selected from the group of substances consisting of gargled fluid, saliva not swabbed from the throat of a patient, nasal wash, nasal aspirate, throat wash, oral wash, spit, material (liquid and/or solid) from (a) (i) a nasal swab or (ii) a nasopharyngeal swab mixed with (b) (i) the gargled fluid and/or (ii) the nasal wash, and/or any combination of the above fluid samples.

For some applications, testing device 20 further comprises a filter chamber 36 that is (a) disposed downstream of liquid container 30, (b) shaped so as to define an inlet 38, and (c) in fluid communication with filter 32. Filter chamber 36 is shaped such that when filter 32 is pushed into the filter chamber, such as described hereinbelow with reference to FIGS. 1D-E, the filter chamber collects filter 32 into a relatively small volume, thereby increasing the consolidated sensitivity of tests for detecting particulate trapped by filter 32. If, by contrast, filter 32 were flat in liquid container 30, it would be difficult to collect a sample from a high percentage of the surface of the filter. In addition, filter chamber 36 readily hosts at least one extraction reagent 86 and a test strip 88, as described hereinbelow with reference to FIGS. 1G-H.

Optionally, filter chamber 36 is nipple-shaped. For some applications in which testing device 20 comprises waste liquid receptacle 46, filter chamber 36 is laterally surrounded by at least a portion of waste liquid receptacle 46, such as shown in FIGS. 1A-H. Alternatively or additionally, for some applications, filter chamber 36 is disposed within waste liquid receptacle 46, such as shown in FIGS. 1A-H.

For some applications, such as shown in FIGS. 1A-H, filter chamber 36 comprises one or more pressure-activated valves 50, not disposed at inlet 38. For applications in which testing device 20 comprises waste liquid receptacle 46, the one or more pressure-activated valves 50 are typically disposed in fluid communication between filter chamber 36 and waste liquid receptacle 46. For some applications, liquid container 30 is shaped so as to define one or more openings 51 (typically, non-valved openings) through a wall of liquid container 30, the one or more openings 51 are downstream of filter 32 when filter 32 is removably disposed upstream of filter chamber 36 with filter 32 partially covering inlet 38, and filter chamber 36 is not disposed so as to receive liquid 22 that is driven through the one or more openings 51. The one or more openings 51 allow liquid 22 to pass, thereby drawing the liquid through filter 32. (As described hereinabove with reference to FIGS. 1A-B, testing device 20 may comprise an upstream component 70 and a downstream component 72 that are removably coupled together; in such configurations, the one or more openings 51 defined by liquid container 30 may optionally be defined by the portion of downstream component 72 that helps define liquid container 30.)

For some applications, such as shown in FIGS. 1A-D, filter 32 is removably disposed upstream of filter chamber 36 with filter 32 partially covering inlet 38. For some applications, inlet 38 has an inlet centroid 52 that is disposed within a distance of a filter centroid 54, the distance equal to 50% of a greatest dimension of filter 32, when filter 32 is removably disposed upstream of filter chamber 36 with filter 32 partially covering inlet 38. For example, filter 32 may be centered upstream of inlet 38.

For some applications, an elongate member 56 is provided that configured to push at least a portion of filter 32 into filter chamber 36. Optionally, elongate member 56 comprises a swab 58 at a distal end of the elongate member. In applications in which filter chamber 36 comprises one or more pressure-activated valves 50, inserting elongate member 56 into filter chamber may squeeze any liquid 22 remaining in filter chamber 36 through one or more pressure-activated valves 50 and out of filter chamber 36. For other applications in which liquid-pressure source 34 comprises plunger 40, plunger head 42 is configured to push at least a portion of filter 32 into filter chamber 36 (configuration not shown). In applications in which filter chamber 36 comprises one or more pressure-activated valves 50, inserting plunger head 42 into filter chamber may squeeze any liquid 22 remaining in filter chamber 36 through one or more pressure-activated valves 50 and out of filter chamber 36.

Reference is still made to FIGS. 1A-H. In an application of the present invention, testing device 20 further comprises one or more valves 60. For these applications, filter 32 is typically disposed in or downstream of liquid container 30 and upstream of the one or more valves 60. Liquid-pressure source 34 is arranged to apply pressure to drive liquid 22 contained in liquid container 30 through filter 32 and then through the one or more valves 60. For applications in which testing device 20 comprises waste liquid receptacle 46, waste liquid receptacle 46 is typically coupled (removably or permanently) to liquid container 30 downstream of the one or more valves 60, and liquid-pressure source 34 is arranged to apply pressure to drive liquid 22 contained in liquid container 30 through filter 32, then through the one or more valves 60, and then into waste liquid receptacle 46. Typically filter chamber 36 is not disposed so as to receive liquid 22 that is driven through at least one of the one or more valves 60.

For some applications, the one or more valves 60 comprise one or more pressure-activated valves. For example, as mentioned above, filter chamber 36 may comprise one or more pressure-activated valves 50, not disposed at inlet 38. For example, the pressure-activated valves may be formed from slits or flaps in an elastic material (such as silicone), or may comprise any small valves known in the valve art. The one or more pressure-activated valves are configured to open at the higher pressure applied by liquid-pressure source 34, so as to allow liquid 22 to pass through filter 32, and to remain closed at the much lower pressure applied by at least one extraction reagent 86, as described hereinbelow with reference to FIGS. 1G-H. Preventing the leakage of the at least one extraction reagent 86 causes the at least one extraction reagent 86 to bathe filter 32, which is beneficial for optimal testing for particulate trapped by filter 32 using a test strip 88, also as described hereinbelow with reference to FIGS. 1G-H.

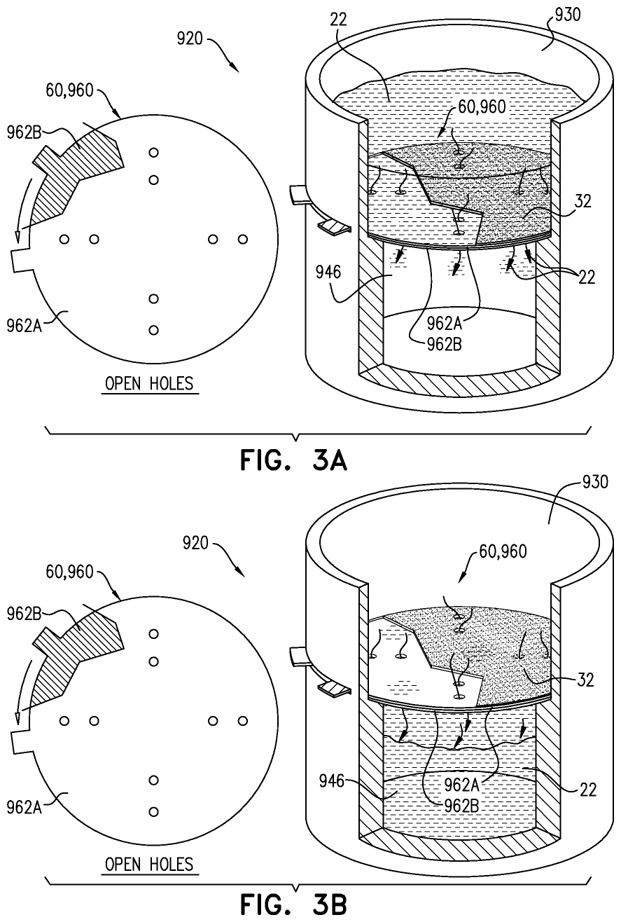

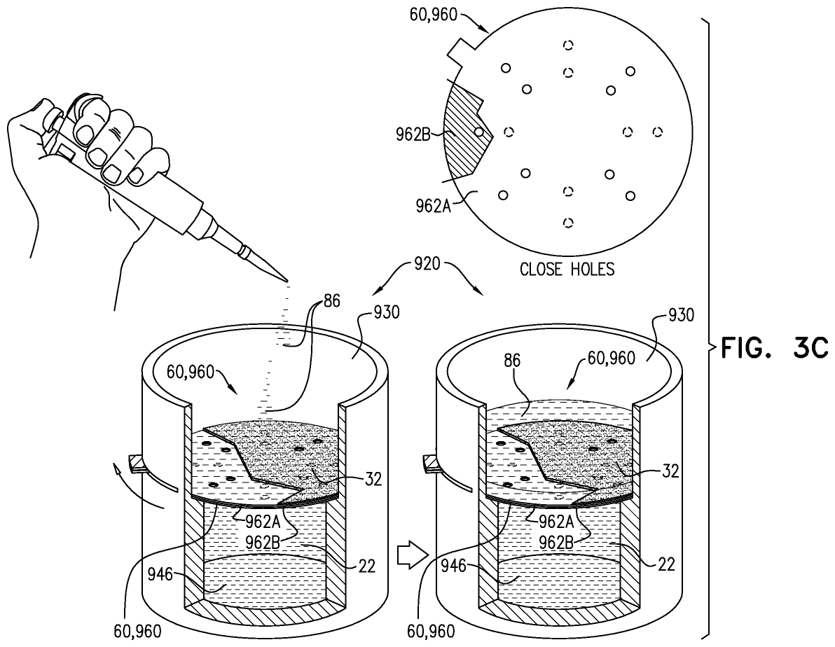

Alternatively or additionally, for some applications, the one or more valves 60 comprise one or more non-pressure-activated valves, such as described hereinbelow with reference to FIGS. 3A-C, 4A-H, 5A-B, 6A-B, and/or 7A-B.

For some applications, sterile packaging is provided, in which at least liquid container 30, filter chamber 36, the one or more valves 60, and/or filter 32 are removably disposed. The sterile packaging comprises one or more sterile packages; for example, each element may be removably disposed in a separate one of the packages, and/or more than one the elements may be disposed in a single one of the packages.

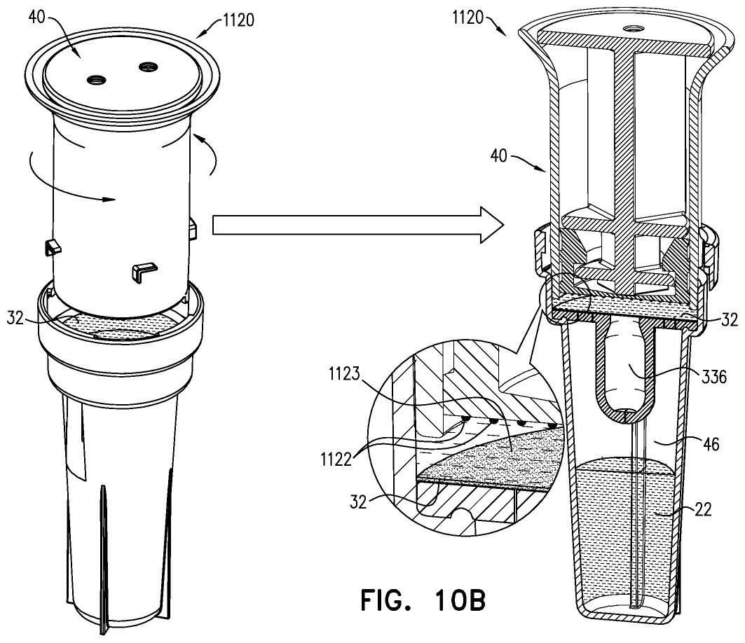

For some applications, at least one container comprising the at least one extraction reagent 86 is provided. For example, extraction reagent 86 may comprise any of the extraction reagents described at extraction step 1804 of the method described hereinbelow with reference to FIG. 11. For applications in which more than one extraction reagent 86 is provided, and/or extraction reagent 86 comprises a plurality of substances, each of the extraction reagents 86 and/or substances may be provided in a separate container, and the extraction reagents 86 and/or substances are combined prior to (e g , immediately prior to) performing the assay. Alternatively or additionally, for some applications, a test strip 88 is provided. Typically, test strip 88 is a lateral flow test strip, such as a lateral flow immunoassay (e.g., chromatographic immunoassay) test strip, as is known in the art. For example, test strip 88 may contain an antibody specific to an antigen, and the mixture migrates up the test strip and reacts with the antibody, thus generating a line on the test strip; the presence of this line indicates a positive test result. Alternatively or additionally, for some applications, a container is provided containing a solution for use in a detecting a pathogen.

Reference is still made to FIGS. 1A-H. In an application of the present invention, testing device 20 comprises an upstream component 70 and a downstream component 72 (labeled in FIGS. 1B and 1C).

Upstream component 70 typically comprises:
a plunger housing 74, which is shaped so as to define an upstream opening 76 (labeled in FIG. 1B) and a downstream opening 78 (labeled in FIG. 1C); and
plunger 40, which comprises a downstream plunger head 42 that is shaped so as to be insertable into plunger housing 74 so as to form a movable seal with a wall of plunger housing 74; typically, an area of a downstream surface 80 of downstream plunger head 42 equals between 80% and 100% of an area of downstream opening 78 (unlike in conventional syringes, in which the downstream surface of the plunger head is typically much larger than the narrow downstream opening of the syringe barrel).

Typically, plunger housing 74 does not comprise a Luer lock or any other type of needle-coupling mechanism.

Downstream component 72 typically comprises:
filter 32, which has a filter surface area if an upstream side of the filter equal to at least 80% of the area of downstream surface 80 of the downstream plunger head 42;
waste liquid receptacle 46, disposed downstream of filter 32; and
for applications in which it is provide, filter chamber 36.

Testing device 20 is shaped so as to define liquid container 30 for containing liquid 22. Upstream component 70 and downstream component 72 are configured to be removably coupled together so as to form a liquid-impermeable seal, as shown in FIGS. 1A and 1B. FIG. 1C shows upstream component 70 and downstream component 72 after they have been decoupled from each other. For example, upstream component 70 and downstream component 72 may be configured to be removably coupled together by click-fitting together, by friction-fitting together, by twist-and-lock fitting together, or by magnetic coupling together.

For some applications, such as shown in FIGS. 1A-B, upstream component 70 and downstream component 72 are configured to be removably coupled together so as to form the liquid-impermeable seal, such that upstream component 70 and downstream component 72 partially overlap each other at an axial overlap region 82 (labeled in FIG. 1B) that at least partially defines liquid container 30. For other applications, upstream component 70 and downstream component 72 do not axially overlap (configuration not shown); in these other applications, liquid container 30 is optionally defined only by downstream component 72 and not by upstream component 70. For some applications, as perhaps best shown in the blow-up in FIG. 1A, an outer edge of filter 32 is squeezed directly or indirectly between upstream component 70 and downstream component 72 to hold the filter in place until upstream component 70 is decoupled from downstream component 72.

In general, in all of the configurations of testing devices described herein that comprise upstream and downstream components that are removably coupled together, the liquid container may be defined in part by the upstream component and in part by the downstream component. For example, a distal downstream wall of the liquid container that supports the filter may be defined by the downstream component, while the lateral wall of the liquid container may be defined by the upstream component or by the upstream and downstream components in combination.

For some applications, such as shown in FIGS. 1C-D, testing device 20 is configured such that at least 80% of the surface area of an upstream side of filter 32 is exposed to outside testing device 20 when upstream component 70 and downstream component 72 are decoupled from each other.

For some applications, an area of upstream opening 76 is greater than the area of downstream opening 78. For example, a diameter of upstream opening 76 may be at least 10% (e.g., 20%, such as 30%) greater than a diameter of downstream opening 78. For some of these applications, plunger housing 74 includes an upstream end portion 84 (labeled in FIG. 1B) that includes upstream opening 76, and upstream end portion 84 is conical and/or funnel-shaped.

Reference is still made to FIGS. 1A-H. In an application of the present invention, a method is provided for testing liquid 22, e.g., a non-centrifuged oronasopharyngeal fluid sample, for the presence of the particulate. For some applications, the particulate comprises biological particulate, for example, a virus (e.g., a free virus and/or a viral particle within a biological cell), a cellular biomarker (e.g., a tumor-specific cellular biomarker, an inflammatory-specific cellular biomarker, and/or a disease-specific cellular biomarker), a biological cell (e.g., cells containing a virus and/or a cellular biomarker, such as epithelial cells or leukocytes), a biological antigen (e.g., a protein antigen or a carbohydrate antigen that is a viral target or a cellular biomarker), and/or a nucleic acid (e.g., DNA or RNA, such as a viral DNA target or a viral RNA target).

For applications in which one or more components of testing device 20 are removably disposed in sterile packing, the one or more components are removed from the sterile packaging.

As shown in FIG. 1A, the method comprises receiving, in liquid container 30, liquid 22 from a patient's mouth. For some applications, liquid 22 comprises gargled fluid, i.e., a gargle fluid that the patient has gargled in his or her mouth and spit out, perhaps along with some saliva. In the present application, including in the claims and Inventive Concepts, “gargled fluid” means “gargle fluid” that has been gargled by a patient. Typically, the gargle fluid includes water, carbonated water, saline (e.g., phosphate buffered saline), pelargonium sidoides extract, tannic acid, balloon flower platycodon grandiflorus, berberine sulfate, S-carboxymethylcysteine, curcumin, coloring, flavoring, a detergent (such as Polysorbate 20 (e.g., Tween® 20)), or any combination thereof In some applications, the gargle fluid is carbonated. Alternatively or additionally, for some applications, a detergent, such as Polysorbate 20 (e.g., Tween® 20) is added to the gargled fluid after being gargled by the patient. Alternatively, liquid 22 may comprise another type of biological fluid, such as blood (e.g., diluted blood), urine, stool (e.g., diluted stool), gastrointestinal (GI) fluid, or bronchoalveolar lavage fluid.

Alternatively, liquid 22 comprises saliva not swabbed from the throat of a patient (i.e., the saliva was collected without swabbing the patient's throat). (The distinction between “swab” as a verb and as a noun is noted. A “swab” (as a noun) may be used to obtain saliva without “swabbing” (as a verb) the patient's throat. For example, the patient may suck on a swab, or a swab may be dipped in a container into which gargle fluid or saliva has been placed.) By contrast, in commonly-practiced techniques for testing for Influenza and for testing for SARS-CoV-2, the nasal cavity and/or the nasopharynx is swabbed.

Liquid 22 (e.g., gargled fluid or saliva) may be spit directly by the patient into liquid container 30 or transferred by a healthcare worker from another container into which the patient spit. Alternatively, in the case of saliva, the saliva may be collected from the patient's mouth by having the patient suck on a swab or other absorbent collecting element, such as flocked swabs or cotton rolls.

For applications in which testing device 20 comprises plunger 40 and plunger housing 74, such as described above, liquid 22 is typically received in liquid container 30 before plunger 40 has been inserted into plunger housing 74 (or liquid container 30).

As shown in FIG. 1B, pressure is applied to drive liquid 22 contained in liquid container 30 of testing device 20 through filter 32, such as using one or more of the techniques for applying pressure described hereinabove. For applications in which testing device 20 comprises the one or more valves 60, the pressure also drives liquid 22 through the one or more valves 60 after the liquid is driven through filter 32. For applications in which liquid container 30 is shaped so as to define the one or more openings 51, as described hereinabove, the pressure drives liquid 22 through the one or more openings 51. Typically, toward the end of the application of the pressure, some air trapped in liquid container 30 is blown through filter 32, helping to expel most of liquid 22 remaining in filter chamber 36 and generally dry the filter chamber. For applications in which testing device 20 comprises waste liquid receptacle 46, applying the pressure drives liquid 22 contained in liquid container 30 through filter 32, then through the one or more valves 60, and then into waste liquid receptacle 46. For some applications in which testing device 20 comprises filter chamber 36, applying the pressure also drives some of liquid 22 into filter chamber 36. For some applications, testing device 20 further comprises a release button that pushes on filter chamber 36 to extract any remaining gargled fluid upon completion of application of the pressure (configuration not shown).

As shown in FIG. 1C, for applications in which testing device 20 comprises upstream component 70 and downstream component 72, upstream component 70 is decoupled from downstream component 72, in order to expose and provide access to filter 32. Instead removing plunger 40 from liquid container 30 might cause some of liquid 22 to spray out of liquid container.

As shown in FIG. 1A-D, for some application in which testing device 20 comprises filter chamber 36, the pressure is applied while filter 32 is removably disposed upstream of filter chamber 36 with filter 32 partially covering inlet 38. For some of these applications, after applying the pressure and before testing for the presence of the particulate trapped by filter 32, at least a portion of filter 32 is pushed into filter chamber 36, such as shown in FIGS. 1D-F. For example, the at least a portion of filter 32 may be pushed into filter chamber 36 using an elongate member 56, such as shown in FIG. 1D-E, using plunger head 42 (configuration not shown), or using gas pressure and/or suction (configuration not shown). For applications in which filter chamber 36 comprises one or more pressure-activated valves 50, such as described hereinabove, the elongate member 56 (e.g., swab 58 thereof) drives liquid 22 in filter chamber 36 out of filter chamber 36 through the one or more pressure-activated valves 50, into waste liquid receptacle 46 if provided, such as shown in FIG. 1E.

As shown in FIGS. 1E-F, for some applications, a sample is taken from filter 32 (either from a surface of the filter or of the filter itself, such as a small part of the filter) using elongate member 56 (e.g., swab 58 thereof), and the sample is tested, outside testing device 20, for the presence of the particulate, using the techniques described hereinbelow with reference to FIG. 11. The testing may be performed by placing the sample (optionally while still on swab 58) into a test tube 85 containing extraction reagent 86; for example, extraction reagent 86 may comprise any of the extraction reagents described at extraction step 1804 of the method described hereinbelow with reference to FIG. 11.

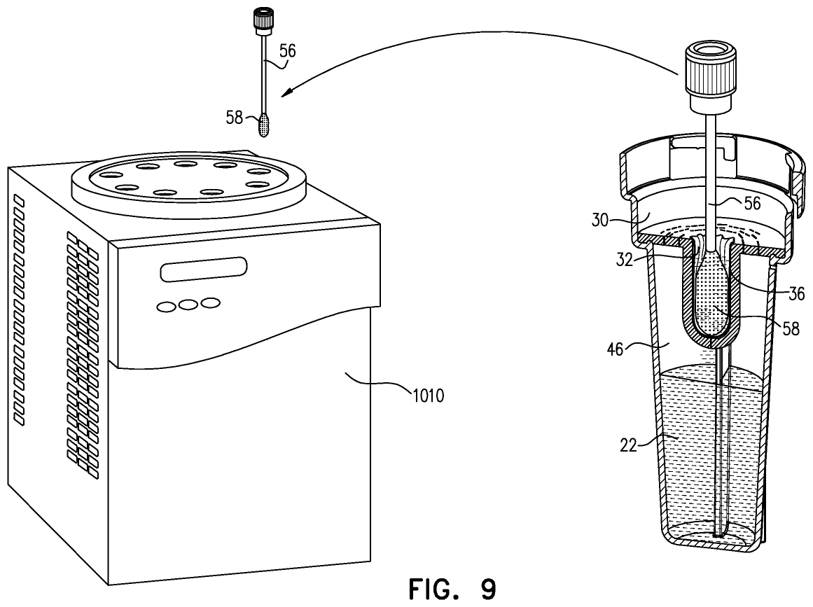

Alternatively, for some applications, the entire filter 32 is removed from testing device 20 and tested, outside testing device 20, for the presence of the particulate, for example, using any of the testing techniques described hereinbelow with reference to FIG. 9 regarding external analysis device 1010. For some applications, the entire filter 32 is removed from testing device 20 and transferred into a transport liquid before testing, outside testing device 20, for the presence of the particulate. For some applications, the entire filter 32 is removed from testing device 20 and transferred into extraction reagent 86 before testing, outside testing device 20, for the presence of the particulate.

As shown in FIGS. 1G-H, the method further comprises testing, within testing device 20, for the presence of a particulate, such as a virus or a cellular biomarker, trapped by filter 32 while filter 32 is disposed in testing device 20. For applications in which testing device 20 comprises the one or more valves 60, the testing is performed while the one or more valves 60 are closed. Alternatively or additionally, for applications in which testing device 20 comprises filter chamber 36, the testing is performed within filter chamber 36 while filter 32 is disposed at least partially in the filter chamber.

For some applications, the testing for the presence of the particulate is performed by:
applying an extraction reagent 86 to filter 32, such as shown in FIG. 1G; for example, extraction reagent 86 may comprise any of the extraction reagents described at extraction step 1804 of the method described hereinbelow with reference to FIG. 11; for applications in which testing device 20 comprises filter chamber 36, the extraction reagent 86 is typically applied to filter 32 while filter 32 is in filter chamber 36; as mentioned above, for applications in which testing device 20 comprises the one or more valves 60, the testing is performed while the one or more valves 60 are closed so that extraction reagent 86 is retained by filter 32 rather than passing through the filter, and
after applying extraction reagent 86, inserting a test strip 88 into testing device 20 (e.g., into filter chamber 36) and examining the test strip to test for the presence of the particulate, such as a virus or a cellular biomarker, such as shown in FIG. 1H; optionally, filter 32 is mixed after application of extraction reagent 86 but before insertion of test strip 88.

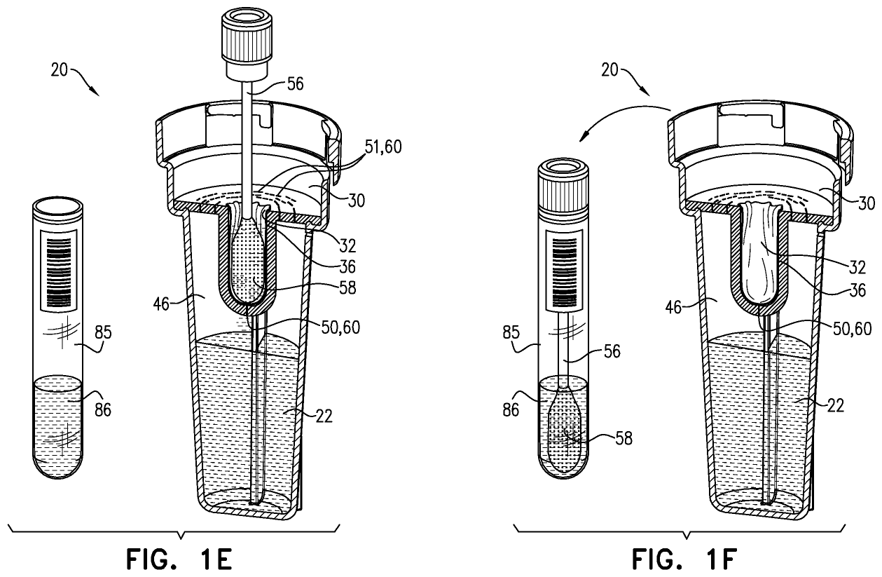

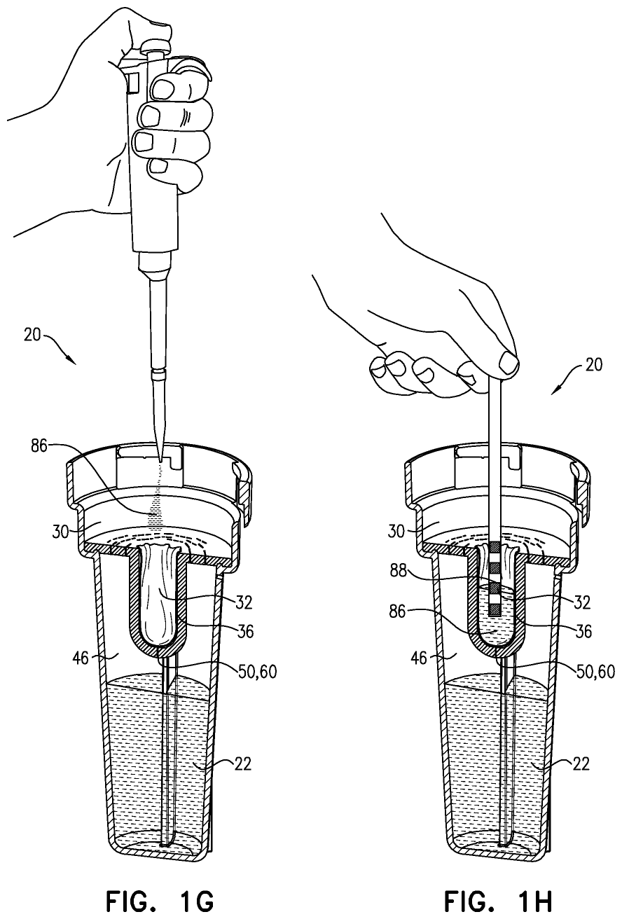

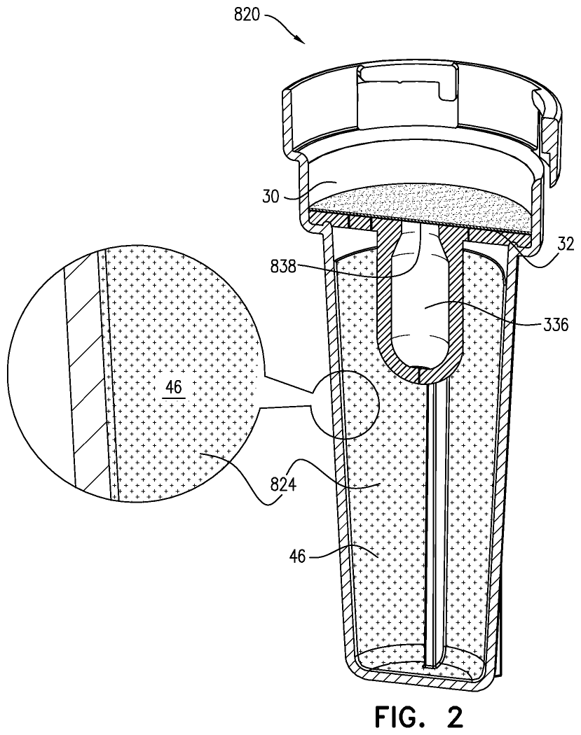

Reference is now made to FIG. 2, which is a schematic illustration of a testing device 820 for testing for presence of a particulate, such as a virus or a cellular biomarker, in liquid 22, such as oronasopharyngeal fluid, in accordance with an application of the present invention. Other than as described below, testing device 820 is similar to the testing devices described hereinabove with reference to FIGS. 1A-H, and may implement any of the features thereof. Similarly, any of testing devices described herein may implement the features of FIG. 2, mutatis mutandis.

Testing device 820 comprises waste liquid receptacle 46, which contains an antimicrobial agent 824, such as a detergent, thiomersal, bleach, or iodine (I/KI) to kill any microbial pathogens that passes through filter 32, to reduce the risk of contamination upon accidental exposure to the liquid in waste liquid receptacle 46.

For some applications, an inlet 838 of a filter chamber 336 of testing device 820 has an inlet area that is less than a greatest cross-sectional area of filter chamber 336, the inlet area and the greatest cross-sectional area measured in respective planes parallel to each other. For example, the inlet area may be no more than 95%, such as no more than 90%, e.g., no more than 80% of the greatest cross-sectional area of filter chamber 336. Providing this narrowing of filter chamber 336 at inlet 838 may help retain filter 32 in filter chamber 336 during withdrawal of elongate member 56, as described hereinabove with reference to FIG. 1F.

Reference is now made to FIGS. 3A-C, which are schematic illustrations of a testing device 920 for testing for presence of a particulate in liquid 22, such as oronasopharyngeal fluid, in accordance with an application of the present invention. (Filter 32 is shown in partial cut-away view to show the one or more non-pressure-activated valves 960 described below.) Other than as described below, testing device 920 is similar to the testing devices described hereinabove with reference to FIGS. 1A-2, and may implement any of the features thereof.

The one or more valves 60 of testing device 920 comprise one or more non-pressure-activated valves 960. For example, the one or more non-pressure-activated valves 960 may be opened and closed by aligning and non-aligning, respectively, sets of openings in two discs 962A and 962B of the one or more non-pressure-activated valves 960, either manually or automatically by the testing device, such as described hereinbelow. Other manual and automated configurations will be readily apparent to those skilled in the art who have read the present application.

During use, liquid 22 is received in a liquid container 930, as shown in FIG. 3A, typically while the one or more non-pressure-activated valves 960 are in an opened state (e.g., with the openings in disc 962A aligned with the openings in disc 962B), thereby allowing liquid 22 to pass through the one or more valves and the filter, optionally into a waste liquid receptacle 946 if provided, as shown in FIGS. 3A-B. Typically, while the one or more non-pressure-activated valves 960 are open, pressure is applied using a liquid-pressure source such as those described herein.

Thereafter, as partially shown in FIG. 3C, the one or more non-pressure-activated valves 960 are closed (e.g., by rotating at least one of discs 962A and 962B so that their respective openings are not aligned with one another) and filter 32 is tested for the presence of particulate trapped by filter 32, such as described hereinabove with reference to FIGS. 1G-H, mutatis mutandis. The closed one or more valves retain extraction reagent 86 in filter 32 by preventing the extraction agent from passing through the filter.

As described hereinabove, for some applications, the testing devices described herein comprise a liquid-pressure source that is arranged to apply pressure to drive liquid contained in the liquid container through the filter and, optionally, then into the waste liquid receptacle. For some of these applications, the testing device is configured to automatically (typically, non-electrically) close one or more non-pressure-activated valves of the testing device after the plunger applies the pressure to drive the liquid contained in the liquid container through the filter and then through the one or more non-pressure-activated valves. For some of these applications, the testing device is configured such that motion of the plunger automatically (typically, non-electrically) closes the one or more non-pressure-activated valves after the plunger applies the pressure to drive the liquid contained in the liquid container through the filter and then through the one or more non-pressure-activated valves. Although the testing device is described in this and the following configurations as non-electrically closing the one or more non-pressure-activated valves, the testing device may alternatively electrically close the one or more non-pressure-activated valves, such as using a motor.

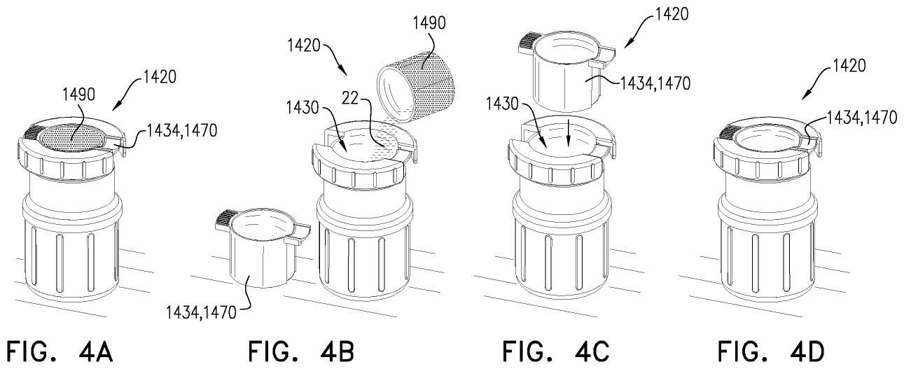

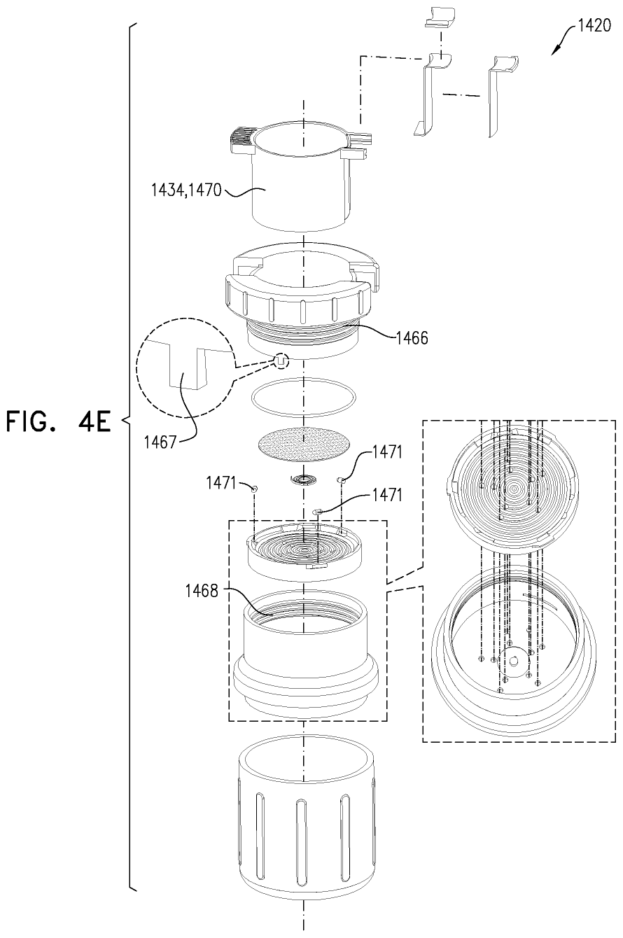

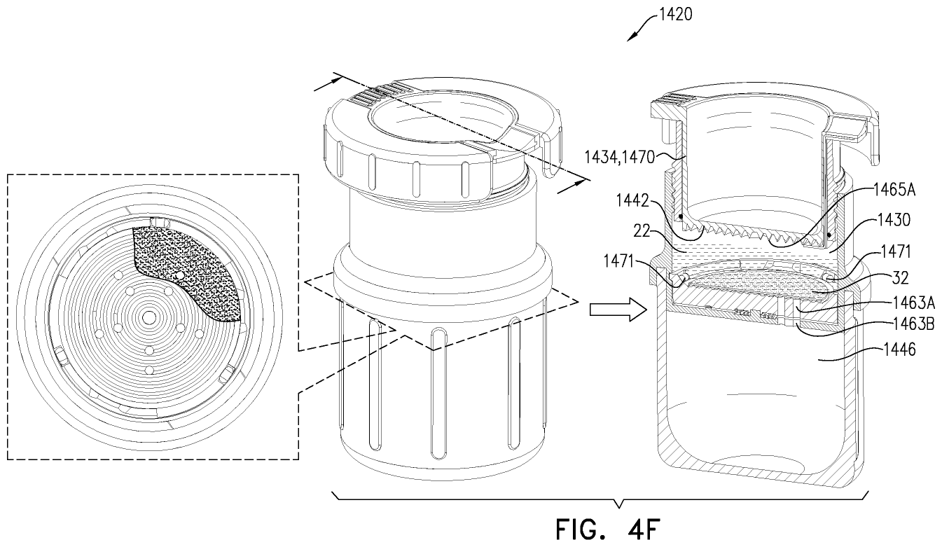

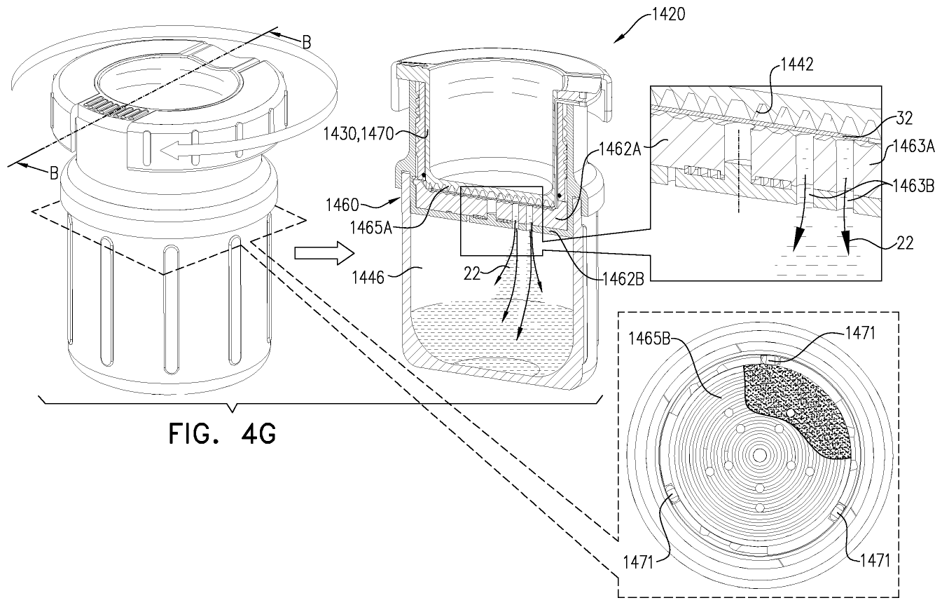

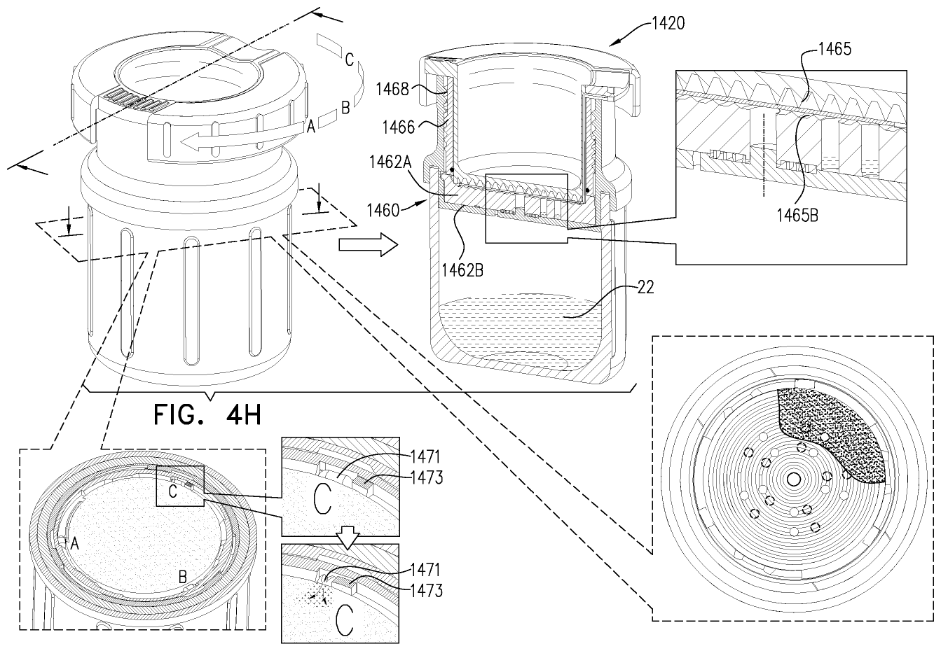

Reference is now made to FIGS. 4A-H, which are schematic illustrations of a testing device 1420 for testing for presence of a particulate, such as a virus or a cellular biomarker, in liquid 22, such as oronasopharyngeal fluid, in accordance with an application of the present invention. Other than as described below, testing device 1420 is similar to the testing devices described hereinabove with reference to FIGS. 1A-2 and 3A-C, and may implement any of the features thereof, mutatis mutandis. Testing device 1420 comprises a liquid-pressure source 1434, which comprises a plunger 1440, which comprises a plunger head 1442 that is shaped so as to be insertable into a liquid container 1430. Testing device 1420 typically further comprises a waste liquid receptacle 1446, which is coupled to liquid container 1430 downstream of filter 32. Plunger 1440 is arranged to apply pressure to drive liquid 22 contained in liquid container 1430 through filter 32 and then through one or more non-pressure-activated valves 1460 of testing device 1420, and into waste liquid receptacle 1446, if provided, as shown in FIGS. 4F-G.

As shown in FIG. 4H, testing device 1420 is configured such that rotational motion of plunger 1440 automatically (typically, non-electrically) closes the one or more non-pressure-activated valves 1460 of testing device 1420 after plunger 1440 applies the pressure to drive liquid 22 contained in liquid container 1430 through filter 32 and then through the one or more non-pressure-activated valves 1460. For example, the last turn of plunger 1440, or a fraction of the last turn (which may or may not include the last portion of the last turn), may automatically close the one or more non-pressure-activated valves 1460.

For some applications, plunger 1440 is shaped so as to define one or more plunger threads 1466, and an internal wall of liquid container 1430 is shaped so as to define one or more liquid-container threads 1468 that engage the one or more plunger threads 1466 such that rotation of plunger 1440 advances plunger 1440 in a downstream direction within liquid container 1430. Advancing plunger 1440 helps control the speed of the advancement and helps maintain steady advancement against pressure in liquid container 1430.

For some applications, the one or more non-pressure-activated valves 1460 comprise two discs 1462A and 1462B, which are shaped so as to define respective sets of openings 1463A and 1463B, for example as described hereinabove with reference to FIGS. 3A-C. For these applications, testing device 1420 is configured such that rotational motion of plunger 1440 automatically closes the one or more non-pressure-activated valves 1460 by rotating at least one of the two discs 1462A and 1462B with respect to the other of the discs, after plunger 1440 applies the pressure to drive liquid 22 contained in liquid container 1430 through filter 32 and then through the one or more non-pressure-activated valves 1460. For example, the last turn of plunger 1440, or a fraction of the last turn (which may or may not include the last portion of the last turn), may automatically rotate the at least one of the discs. For example, ridges 1465A on plunger head 1442 may engage, via filter 32, corresponding ridges 1465B on an upstream surface of disc 1462A after plunger 1440 has been advanced in a downstream direction into contact with disc 1462A. Alternatively or additionally, for some applications, testing device 1420 comprises one or more tabs 1467 that rotate the upper disc and/or break the capsules described hereinbelow.

For some applications, testing device 1420 comprises one or more reagent containers 1471, such as capsules, that contain one or more extraction reagents 86 (either the same type of extraction reagents or differing extraction reagents). Reagent containers 1471 are disposed at least partially in liquid container 1430, such that upon opening of the containers, such as by crushing, tearing, or breaking, extraction reagents 86 are released into liquid container 1430, typically near filter 32. For example, testing device 1420 may configured such that rotational motion of plunger 1440 automatically opens reagents containers 1471, such as by bringing one or more respective protrusions 1473 into contact with the reagent containers. For example, a fraction of the last turn (or the last turn), may automatically open reagents containers 1471. Typically, a fraction of last turn (may or may not include the last portion of the last turn) opens reagents containers 1471, and the fraction occurs after the fraction of the last turn that closes the one or more non-pressure-activated valves 1460, such that the one or more valves are closed before the reagents are released.

Reference is made to FIGS. 4A-D, which illustrate a portion of a method for using testing device 1420 for testing liquid 22, such as oronasopharyngeal fluid, for the presence of the particulate. This method is optional, and testing device 1420 is not necessarily used in this manner and thus does not necessarily comprise the elements necessary for use in this manner. These techniques may be also be practiced in combination with any of the testing devices described herein for which they are applicable, mutatis mutandis.

In this portion of the method, the user typically receives testing device 1420 with the elements thereof removably coupled together, as shown in FIG. 4A. The user removes plunger 1440 of liquid-pressure source 1434 and a container 1490 from the body of testing device 1420, as shown in FIG. 4B; for example, plunger 1440 may be removed from liquid container 1430 and container 1490 may be removed from a cavity defined by plunger 1440. Liquid 22 is received from the patient into container 1490 (step not shown). Liquid 22 is poured from container 1490 into liquid container 1430, as shown in FIG. 4B. Plunger 1440 is reinserted into liquid container 1430, as shown in FIG. 4D.

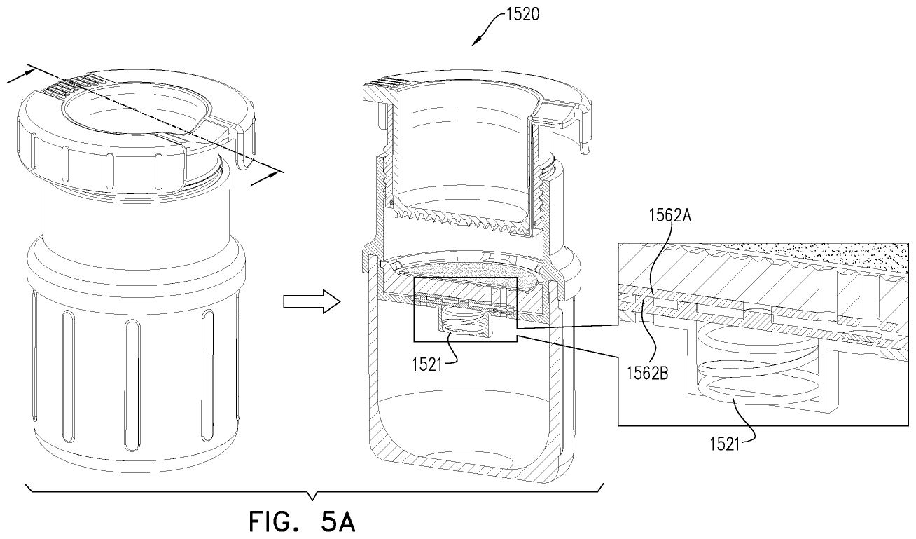

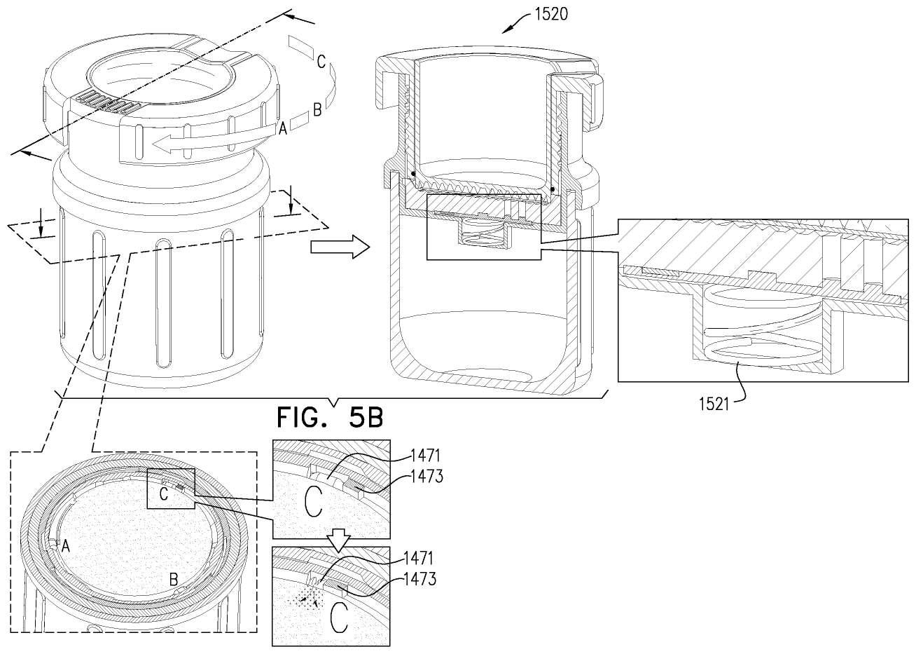

Reference is now made to FIGS. 5A-B, which are schematic illustrations of a testing device 1520 for testing for presence of a particulate, such as a virus or a cellular biomarker, in liquid 22, such as oronasopharyngeal fluid, in accordance with an application of the present invention. Other than as described below, testing device 1520 is similar to testing device 1420 described hereinabove with reference to FIGS. 4A-H. and may implement any of the features thereof, mutatis mutandis. Testing device 1520 comprises a spring 1521, which is biased to hold slightly separated discs 1562A and 1562B of one or more non-pressure-activated valves 1560 of testing device 1520, thereby creating a fluid flow path through the openings of the discs, as shown in FIG. 5A. The downstream advancing of the plunger pushes the upper disc 1562A downstream and thus the discs together (and compresses the spring), as shown in FIG. 5B, thereby blocking fluid flow through the openings. The discs typically do not rotate with respect to one another in this configuration.

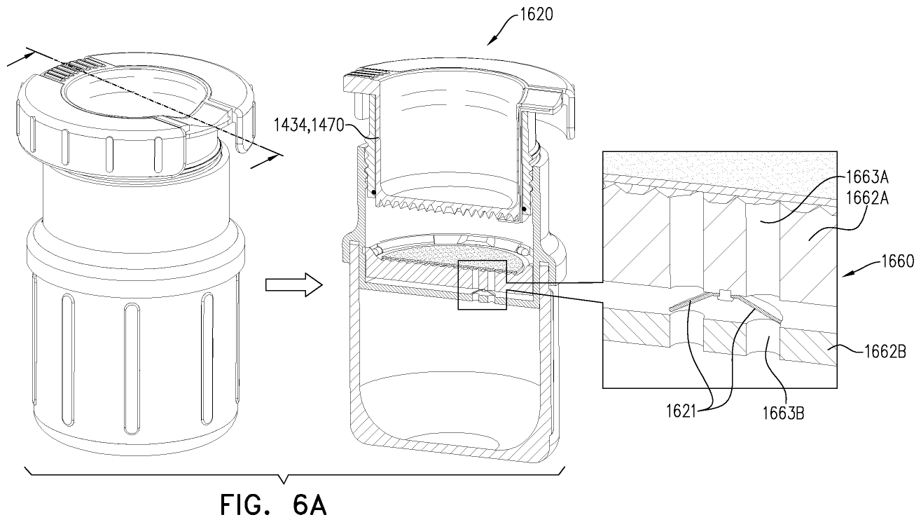

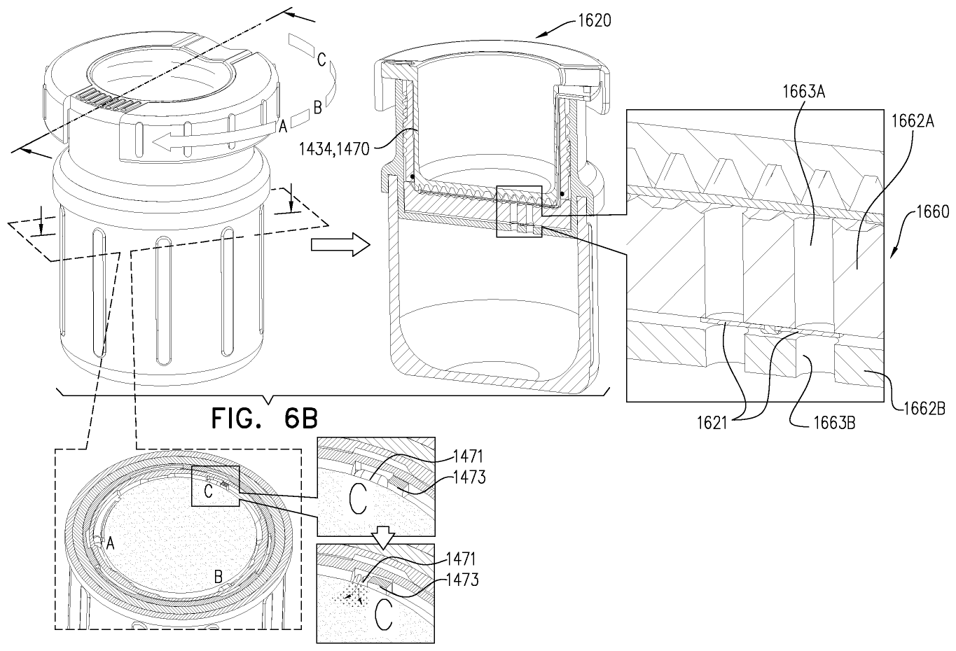

Reference is now made to FIGS. 6A-B, which are schematic illustrations of a testing device 1620 for testing for presence of a particulate, such as a virus or a cellular biomarker, in liquid 22, such as oronasopharyngeal fluid, in accordance with an application of the present invention. Other than as described below, testing device 1620 is similar to testing devices 1420 and 1520 described hereinabove with reference to FIGS. 4A-H and FIGS. 5A-B, respectively, and may implement any of the features thereof, mutatis mutandis. Testing device 1620 comprises one or more flaps 1621, which, in an initial configuration, do not block openings 1663A and 1663B defined by discs 1662A and 1662B, respectively, of one or more non-pressure-activated valves 1660 of testing device 1620, as shown in FIG. 6A. Typically, flaps 1621 are somewhat springy and biased to hold slightly separated discs 1562A and 1562B of one or more non-pressure-activated valves 1560 of testing device 1520. As shown in FIG. 6B, when plunger 1470 is advanced in a downstream direction into contact with disc 1662A, which in turn pushes upstream disc 1662A closer to downstream disc 1662B, thereby causing the one or more flaps 1621 to block openings 1663A and 1663B (such as by displacing or deforming the flaps). The discs typically do not rotate with respect to one another in this configuration.

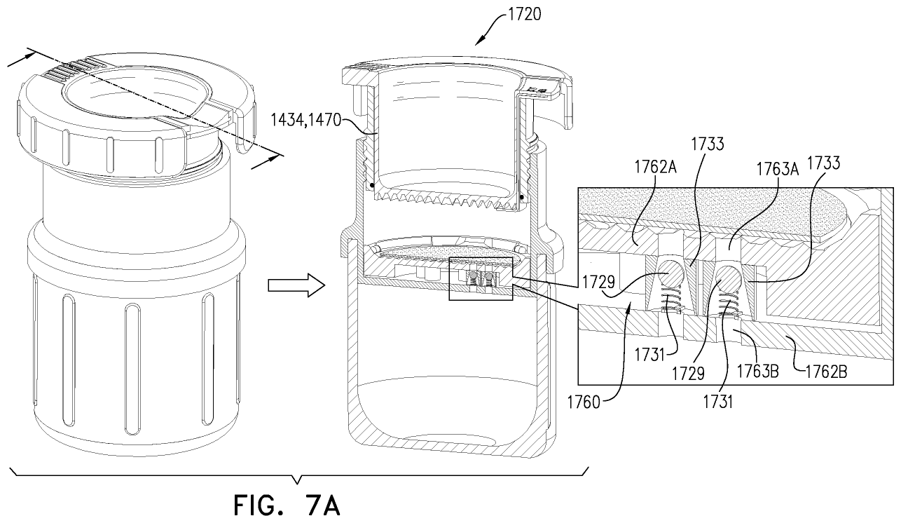

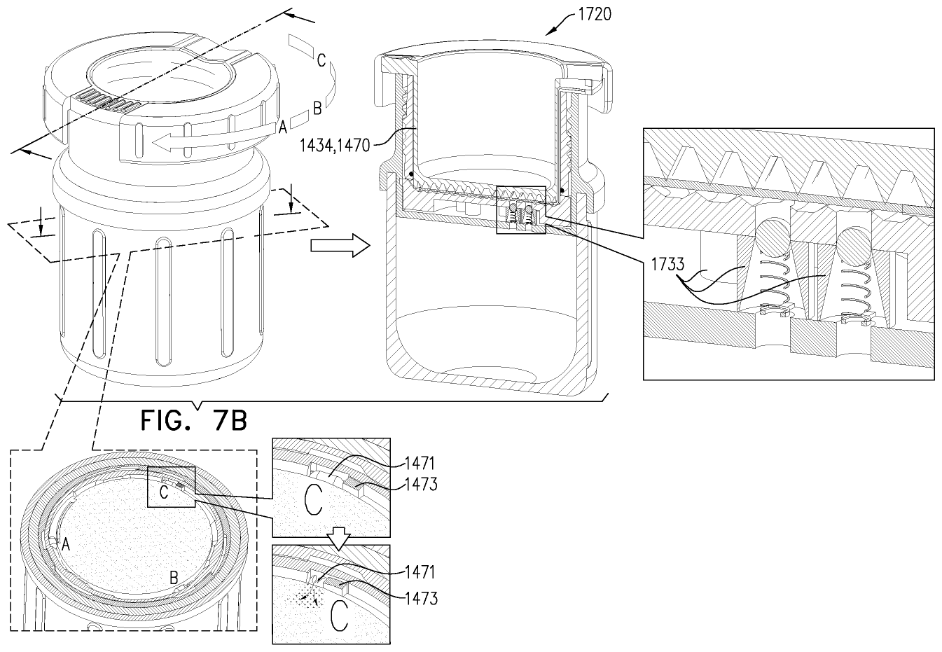

Reference is now made to FIGS. 7A-B, which are schematic illustrations of a testing device 1720 for testing for presence of a particulate, such as a virus or a cellular biomarker, in liquid 22, such as oronasopharyngeal fluid, in accordance with an application of the present invention. Other than as described below, testing device 1720 is similar to testing devices 1420, 1520, and 1620 described hereinabove with reference to FIGS. 4A-H, FIGS. 5A-B, and FIGS. 6A-B, respectively, and may implement any of the features thereof, mutatis mutandis. Testing device 1720 comprises one or more compressible spacers 1733, which hold slightly separated discs 1762A and 1762B of one or more non-pressure-activated valves 1760 of testing device 1720, thereby creating a fluid flow path through the openings of the discs, as shown in FIG. 7A, by holding the discs at a sufficient distance from each other such that one or more plugs 1729 (e.g., spherical plugs) do not plug openings 1763A of upper disc 1762A. The downstream advancing of the plunger pushes upper disc 1762A downstream and thus the discs together (and compresses the compressible spacers 1733), as shown in FIG. 7B, thereby causing the one or more plugs 1729 to block openings 1763A. One or more springs 1731 may be provided to push the one or more plugs 1729 against openings 1763A. Alternatively, a spring similar to spring 1521 of testing device 1520 may be provided instead of or in addition to compressible spacers 1733. The discs typically do not rotate with respect to one another in this configuration.

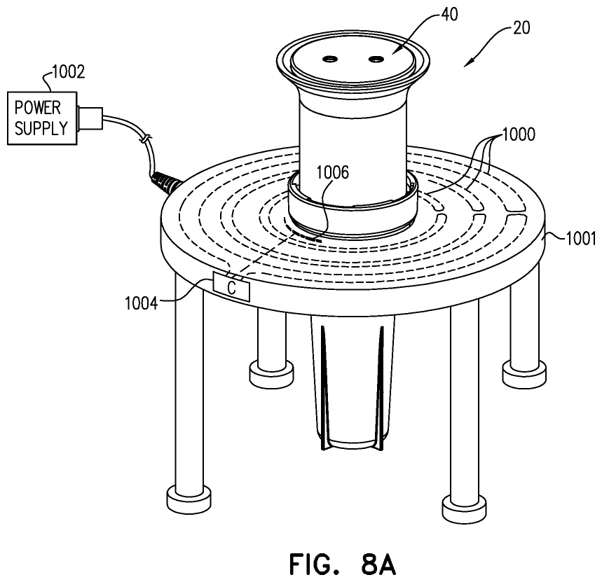

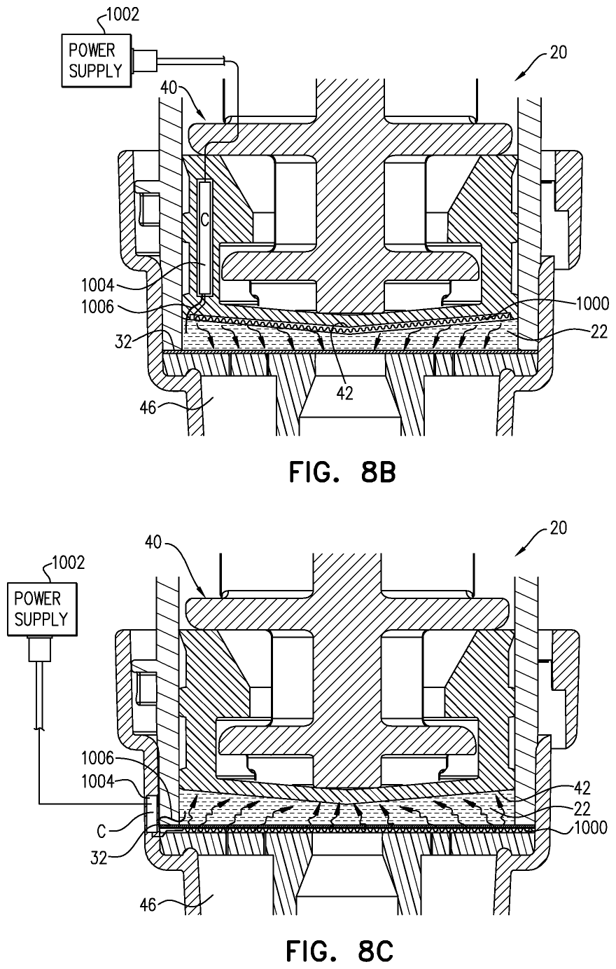

Reference is now made to FIGS. 8A-C, which are schematic illustrations of testing device 20 further comprising one or more heating elements 1000, in accordance with respective applications of the present invention. Although these configurations are illustrated with respect to testing device 20, they may also be combined with the other testing devices described herein, mutatis mutandis. For some applications, these configurations enable heating of liquid 22 and or filter 32 within testing device 20 to extract the cellular components of the cells trapped by filter 32 when preparing the extraction liquid.

In these configurations, testing device 20 further comprises one or more heating elements 1000 that are configured to heat filter 32 and/or liquid 22 in liquid container 30 at a generally constant temperature, typically in the range of 37 and 95 degrees C. (e.g., in the range of 55 and 75 degrees C.), or with multiple heat pulses, typically reaching a maximum temperature in the range of 37 and 95 degrees C. (e.g., in the range of 55 and 75 degrees C.) for each pulse. Heating elements 1000 may comprise, for example, electrical heating elements or chemical heating elements (e.g., a heating bag).

For applications in which heating elements 1000 are electrical, they are coupled in electrical communication with a power supply 1002, such as an external power supply (e.g., the power grid) or an external or internal battery. For example, the coupling may be done using a conventional electrical plug or USB interface. For some applications, testing device comprises control circuitry 1004 and a heat sensor 1006 (e.g., a thermocouple or other thermostat), and control circuitry 1004 is configured to drive heating elements 1000 responsively to a temperature sensed using heat sensor 1006 in order to maintain the generally constant temperature mentioned above.

For some applications, heating elements 1000 are disposed external to main body of testing device 20, such as supported by a stand 1001, such as shown in FIG. 8A.

For other applications, such as shown in FIG. 8B, in which liquid-pressure source 34 comprises plunger 40 that comprises plunger head 42 that is shaped so as to be insertable into liquid container 30, such as described hereinabove, the one or more heating elements 1000 are disposed in the plunger 40, such as in plunger head 42, e.g., separated from the distal end of the plunger head by a layer of material such that liquid 22 does not interfere with the electrical current.

For still other applications, such as shown in FIG. 8C, the one or more heating elements 1000 are disposed downstream of filter 32 (as shown) or upstream of filter 32 (configuration not shown).

For some applications, the one or more heating elements 1000 are configured to heat filter 32 and/or liquid 22 in liquid container 30 after most or nearly all (e.g., at least 90%) of liquid 22 has been driven out of liquid container 30 and the particulate has been trapped by filter 32, such as shown in FIGS. 8B and 8C (the configuration shown in FIG. 8D can alternatively be used with the plunger pushed farther down than illustrated, and the configuration shown in FIG. 8A can also be used).

Reference is now made to FIG. 9, which is a schematic illustration of a method for performing a test, in accordance with an application of the present invention. A sample is taken from filter 32 (either from a surface of the filter or of the filter itself, such as a small part of the filter, and/or such as a small portion of unfiltered liquid remaining above the filter after applying the pressure), e.g., using elongate member 56 (e.g., swab 58 thereof), such as described hereinabove with reference to FIGS. 1E-F. For some applications, filter 32 is entirely removed as the sample. The sample is analyzed using an external analysis device 1010, such as a nucleic acid amplification RST technique, such as isothermal amplification, e.g., using AlereTM i (Abbott Laboratories, Waltham, Massachusetts, USA), or real-time quantitative polymerase chain reaction (qPCR) assaying, typically without first incubating the sample. Alternatively, the sample is cultured and/or incubated (either before placing the sample in external analysis device 1010 or inside device 1010 by device 1010) and, for example, external analysis device 1010 tests the sample using a technique such as a lateral flow immunoassay, an ELISA-based assay, an antibody-coated-beads-based assay, a nucleic-acid-based assay, or a fluorescent immunoassay (FIA).

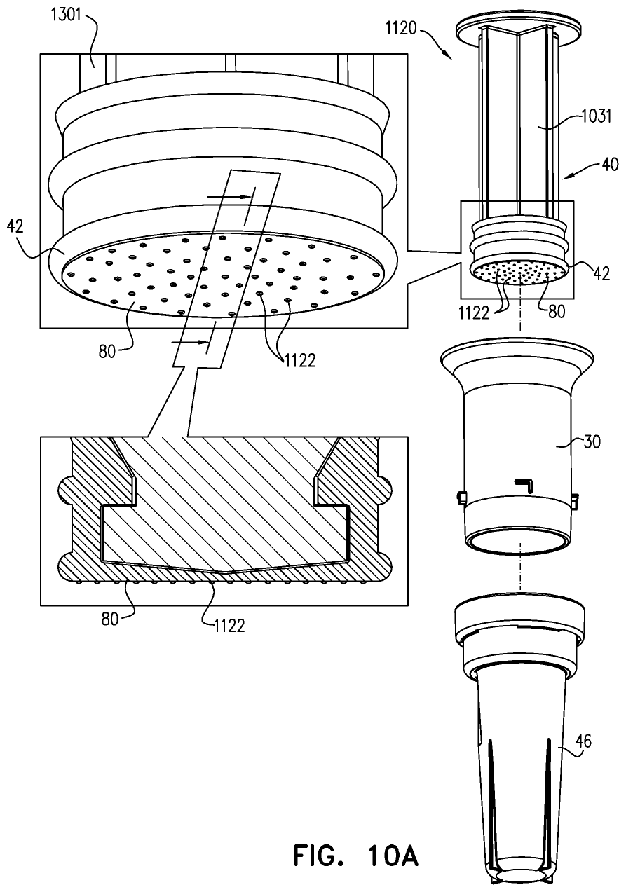

Reference is now made to FIGS. 10A-B, which are schematic illustrations of a method for using a testing device 1120 for testing for presence of a particulate 1123, such as a virus or a cellular biomarker, in liquid 22, such as oronasopharyngeal fluid, in accordance with an application of the present invention. Although testing device 1120 is illustrated as being similar to testing device 20 described hereinabove with reference to FIGS. 1A-H, the method described with reference to testing device 1120 may also be combined with the other testing devices described herein, mutatis mutandis.

Testing device 1120 comprises:
liquid container 30 for containing liquid 22, such as oronasopharyngeal fluid;
filter 32, disposed in or downstream of liquid container 30; and
plunger head 42, which (a) is shaped so as to be insertable into liquid container 30, (b) is configured to apply pressure to drive liquid 22 from liquid container 30 through filter 32, and (c) has downstream surface 80.

In this configuration, downstream surface 80 of plunger head 42 is rough, i.e., is shaped so as to define many small protrusions 1122, such as like sandpaper, or with plastic protrusions, in order to increase the elution efficiency of particulate 1123 by macerating, tearing, or grinding the filter 32.

As shown in FIG. 10B, the method comprises:
pushing plunger head 42 to apply pressure to drive liquid 22 from liquid container 30 of testing device 1120 through filter 32; and
touching downstream surface 80 of plunger head 42 to filter 32.

For some applications, plunger head 42 is rotated while touching downstream surface 80 of plunger head 42 to filter 32 to increase the elution efficiency of particulate 1123 from filter 32 when preparing an extraction liquid, such as by macerating, tearing, or grinding the filter 32. For applications in which downstream surface 80 is decoupled from upstream component 70 by rotation, this rotation may itself increase the elution efficiency of the particulate 1023 from filter 32.

For some applications, touching downstream surface 80 of plunger head 42 to filter 32 comprises grinding filter 32 with rough downstream surface 80.

For some applications, the sample of oral fluid is selected from the group consisting of: gargled fluid gargled by the patient, and saliva not swabbed from a throat of the patient (e.g., spit by the patient, sucked onto a swab by the patient).

For some applications, the sample of oral fluid is collected by drawing the sample oral fluid out of the oral cavity via the mouth by contacting one or more portions of the oral cavity with an absorbent material (e.g., at a tip of a collector shaft), without swabbing the oropharynx. (For example, an ORAcollect®RNA Saliva Collection Device (DNA Genotek Inc., a subsidiary of OraSure Technologies, Inc. (Bethlehem, Pa., USA)) may be used.) For example, the one or more portions of the oral cavity may include one or more of buccal mucosa, the tongue (e.g., under the tongue), the gums (e.g., the lower gums), and/or the palatal mucosa. For example, for swabbing the lower gums, absorbent material (e.g., at a tip of a collector shaft) may be rubbed back and forth along the lower gums several times.

Alternatively, the sample of oral fluid is saliva swabbed from a tonsil of the patient.

Reference is now made to FIG. 11, which is a flowchart depicting a method 1800 for testing a non-centrifuged oronasopharyngeal fluid sample taken from a human or non-human animal for the presence of a particulate, such as a virus or a cellular biomarker, in accordance with some applications of the present invention. Typically, the animal is a mammal, e.g., a human, or a bird. As used in the present application, including the claims and Inventive Concepts, an “animal” includes within its scope both human and non-human animals. Method 1800 may optionally be performed in combination with any of the techniques described hereinabove, including using any of the testing devices described hereinabove, mutatis mutandis, may be performed using conventional filtration device, or may be performed using one or more filters that are not elements of a device.

Typically, liquid 22 taken from the human or non-human animal is a non-centrifuged fluid sample taken from the respiratory system of the human or non-human animal, e.g., one or more non-centrifuged oronasopharyngeal fluid samples taken from one or more of oronasopharyngeal cavities of the human or non-human animal. For some applications, the non-centrifuged oronasopharyngeal fluid sample includes:
a non-centrifuged nasal wash taken from the human or non-human animal,
non-centrifuged nasal aspirate taken from the human or non-human animal,
non-centrifuged gargled fluid taken from the human or non-human animal,
non-centrifuged throat wash taken from the human or non-human animal,
oral wash taken from the human or non-human animal,
material (liquid and/or solid) from (a) (i) a nasal swab or (ii) a nasopharyngeal swab mixed with (b) (i) the non-centrifuged gargled fluid and/or (ii) the nasal wash,
saliva not swabbed from the throat of the human or non-human animal,
spit, and/or
any combination of the above fluid samples, which may increase sensitivity.

Alternatively or additionally, liquid 22 includes, instead of or in addition to the non-centrifuged oronasopharyngeal fluid sample, biological body fluids, such as blood, bronchoalveolar fluid, gastric fluid, urine, cerebrospinal fluid, or cystic fluid, either mixed or not mixed with one or more other fluids, such as saline solution.

Any of the washes described herein may include, in addition to the biological body material, a liquid introduced into the human or non-human animal, such as water, carbonated water, saline (e.g., phosphate buffered saline), pelargonium sidoides extract, tannic acid, balloon flower platycodon grandiflorus, berberine sulfate, S-carboxymethylcysteine, curcumin, coloring, flavoring, a detergent (such as Polysorbate 20 (e.g., Tween® 20)), or any combination thereof

Examples of method 1800 are provided in the experimental data set forth hereinbelow in the section entitled, “Size-Based Filtration Capture of Epithelial Cells from Gargled Fluid and Nasal Wash Followed by Influenza A+B Viral Testing by Rapid Antigen Detection Immunoassay: Two Case Studies.”

At a filtering step 1802, the non-centrifuged oronasopharyngeal fluid sample is passed through one or more porous filters to separate animal cells from the non-centrifuged oronasopharyngeal fluid sample by size-based filtration, using any of the testing devices described herein, a conventional filtration device, or one or more filters that are not elements of a device.

In some applications, at filtering step 1802, the one or more porous filters generally prevent passage of the animal cells while allowing passage of liquid. For example, the passage of liquid may include the passage of particulates smaller than the size of the animal cells, such as particulates that may interfere with testing (e.g., nucleases), such that filtering step 1802 additionally purifies and/or stabilizes the animal cells (e.g., the extracted components of the animal cells), the viral particles (e.g., viral targets, such as a viral antigen, a viral nucleic acid, and a viral nucleoprotein), and/or the cellular biomarkers.

For some applications, the animal cells include one or more types of cell selected from the group consisting of: epithelial cells (squamous cells, columnar cells (e.g., goblet cells), or cuboidal cells) and leukocytes (eosinophils, neutrophils, lymphocytes, monocytes, mast cells, basophils, macrophages, or histiocytes).

For some applications, between 0.5 and 50 cc, such as between 0.5 and 20 cc, such as between 2 and 20 cc, of the non-centrifuged oronasopharyngeal fluid sample is filtered. Alternatively, another amount of the non-centrifuged oronasopharyngeal fluid sample is filtered. Typically, the one or more porous filters have one or more respective pore sizes selected to generally prevent passage of the animal cells while allowing passage of smaller objects. For example, the one or more porous filters may have an average absolute or nominal pore size of between 0.01 and 20 microns, e.g., between 0.03 and 20 microns, such as between 0.1 and 20 microns, e.g., between 0.2 and 20 microns, such as between 1.2 and 20 microns or such as between 0.45 and 9.0 microns, e.g., between 0.45 and 2.0 microns. For example, the average absolute or nominal pore size may be 0.01-0.1 microns, 0.03-0.1 microns, 0.1-2 microns, 0.2-2 microns, 1.2-3 microns, 3-6 microns, 2-10 microns, 6-13 microns, 10-20 microns, or 13-20 microns.

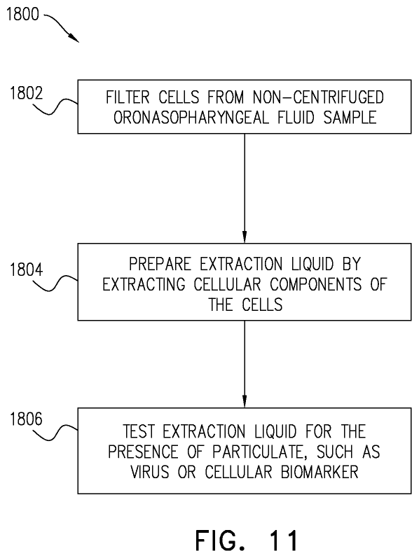

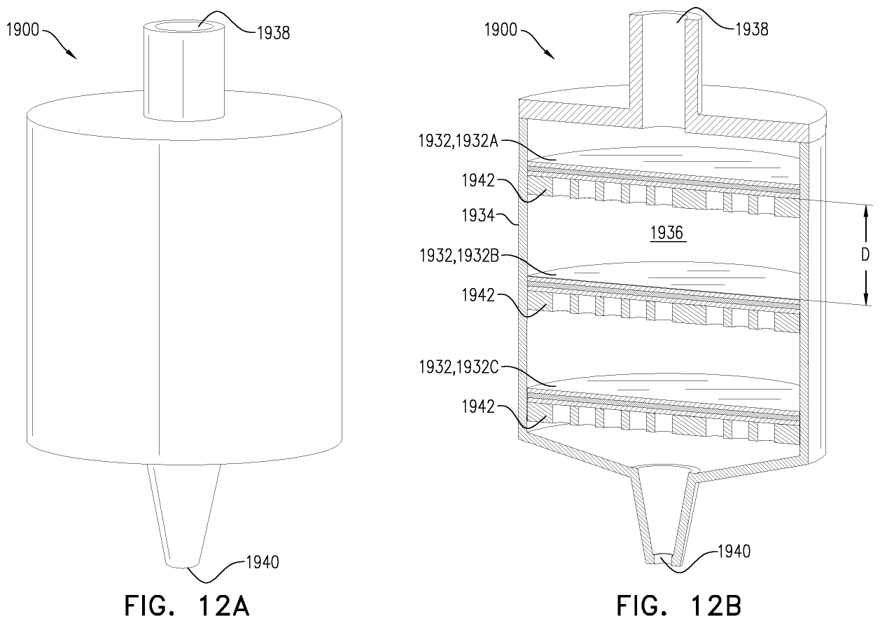

In some applications, at filtering step 1802, the one or more porous filters generally prevent passage of the animal cells and free viral particles while allowing passage of liquid. For example, the passage of liquid may include the passage of particulates smaller than the size of a free viral particles, such as particulates that may interfere with testing (e.g., nucleases), such that filtering step 1802 additionally purifies and/or stabilizes the animal cells (e.g., the extracted components of the animal cells) and/or free viral particles (e.g., viral targets, such as a viral antigen, a viral nucleic acid, and a viral nucleoprotein). In some applications, the non-centrifuged oronasopharyngeal fluid sample is passed through two porous filters, one of which has a pore size selected to generally prevent passage of the animal cells, and the other of which has a pore size selected to generally prevent passage of the free viral particles, for example as described hereinbelow with reference to FIGS. 12A-B. Alternatively or additionally, for some applications, the one or more porous filters have one or more respective pore sizes selected to generally prevent passage of the animal cells by size-based filtration, while simultaneously the one or more porous filters comprise fixed antibodies configured to capture free viral particles by affinity-based filtration. In yet other applications, the filtration step may comprise using multiple porous filters, such that one porous filter has a pore size selected to generally prevent passage of the animal cells by size-based filtration, while one or more porous filters contain fixed antibodies configured to capture free viral particles by affinity-based filtration.

Thereafter, at an extraction step 1804, an extraction liquid is prepared by extracting cellular components of the animal cells separated from the non-centrifuged oronasopharyngeal fluid sample. For some applications, preparing the extraction liquid comprises extracting the virus from the animal cells. For some applications, preparing the extraction liquid comprises exposing an antigen of any of the virus present in the extraction liquid. For some applications, preparing the extraction liquid comprises exposing a nucleic acid of any of the virus present in the extraction liquid. For some applications, preparing the extraction liquid comprises extracting cellular biomarkers from the animal cells. For some applications, preparing the extraction liquid comprises exposing a cellular biomarker present in the extraction liquid.

Thereafter, at a testing step 1806, the extraction liquid is tested for the presence of the particulate, such as a virus or a cellular biomarker. For example, the extraction liquid may be tested by:
performing, on the extraction liquid, an immunoassay, e.g., a lateral flow immunoassay or an enzyme-linked immunosorbent assay (ELISA), such as schematically illustrated in FIG. 1H; or
performing, on the extraction liquid, a molecular-based assay, e.g., nucleic acid hybridization or nucleic acid amplification (such as polymerase chain reaction (PCR) amplification, real-time quantitative PCR (qPCR) amplification, reverse transcriptase PCR (RT-PCR) amplification, and/or isothermal amplification), such as schematically illustrated in FIG. 9.

For some applications, the extraction liquid is tested at testing step 1806 by performing a detection test, such as immunoassay, on the extraction liquid while the extraction liquid is in contact with the one or more filters within a testing device, for example using any of the testing devices described herein, e.g., as schematically illustrated in FIG. 1H. For other applications, the extraction liquid is tested at testing step 1806 by performing a detection test, such as a nucleic acid amplification assay, on the extraction liquid while the extraction liquid is not in contact with the one or more filters outside of the testing device, for example as described hereinabove with reference to FIGS. 1E-F or FIG. 9. For detection tests such as nucleic acid amplification assays, typically the extraction liquid is tested at testing step 1806 outside the testing device, since the testing device that contains the one or more filters generally is too large to fit in standard nucleic acid amplification assays, because the testing device typically is able to accommodate a fluid volume of at least 4 mL and is further typically able to accommodate at least one of the one or more filters that has a surface area equal to at least 2.5 cm2.

For some applications, preparing the extraction liquid at extraction step 1804 comprises extracting and exposing a viral target (such as a viral antigen, a viral nucleic acid, or a viral nucleoprotein), and the extraction liquid is tested for the presence of the virus at testing step 1806 by testing the extraction liquid for the presence of the viral target (such as the viral antigen, the viral nucleic acid, or the viral nucleoprotein, respectively).

In accordance with ordinary usage in the art, testing for the presence of the virus (e.g., by PCR) generally refers to testing for the presence of a portion of the virus, such as a portion of the RNA of the virus, which is considered indicative of the presence of the virus. Accordingly, as referenced herein, extracting the virus from the animal cells generally refers to extracting a portion of the virus from the animal cells, since many enveloped viruses, such as SARS-CoV-2, do not exist as a complete virus inside the animal cells.

For some applications, preparing the extraction liquid at extraction step 1804 comprises extracting and exposing a tumor-specific cellular biomarker (such as a tumor-specific protein, a tumor-specific antibody, a tumor-specific antigen, a tumor-specific enzyme, a tumor-specific peptide, a tumor-specific nucleic acid, a tumor-specific carbohydrate, a tumor-specific hormone, a tumor-specific lipid, a tumor-specific substrate, and/or a tumor-specific metabolite) and the extraction liquid is tested for the presence of the tumor-specific cellular biomarker at testing step 1806 by testing the extraction liquid for the presence of the tumor-specific cellular biomarker.

For some applications, preparing the extraction liquid at extraction step 1804 comprises extracting and exposing an inflammatory-specific cellular biomarker (such as an inflammatory-specific protein, an inflammatory-specific antibody, an inflammatory-specific antigen, an inflammatory-specific enzyme, an inflammatory-specific peptide, an inflammatory-specific nucleic acid, an inflammatory-specific carbohydrate, an inflammatory-specific hormone, an inflammatory-specific lipid, an inflammatory-specific substrate, and/or an inflammatory-specific metabolite) and the extraction liquid is tested for the presence of the inflammatory-specific cellular biomarker at testing step 1806 by testing the extraction liquid for the presence of the inflammatory-specific cellular biomarker.

Some inflammatory-specific cellular biomarkers can act as circumstantial evidence that animal cells specific to a particular form of inflammation may be present or may be present in higher-than-normal levels in the sample and/or in a human or non-human animal from which the sample was taken, enabling differentiation between different forms of inflammation and different causative agents of inflammation, such as an allergic reaction, a bacterial infection, and/or a viral infection. For example, some inflammatory-specific cellular biomarkers, such as high levels of human neutrophil lipocalin and/or calprotectin, that act as circumstantial evidence that neutrophils may be present or may be present in higher-than-normal levels in the sample and/or in a human or non-human animal from which the sample was taken, indicate that the causative agent of the inflammation may be a bacterial infection. In addition, for example, some inflammatory-specific cellular biomarkers, such as high levels of major basic protein (MBP), eosinophil cationic protein (ECP), and/or eosinophil peroxidase (EPX), that act as circumstantial evidence that eosinophils may be present or may be present in higher-than-normal levels in the sample and/or in a human or non-human animal from which the sample was taken, indicate that the causative agent of the inflammation may be an allergy-induced reaction.

For some applications, preparing the extraction liquid at extraction step 1804 comprises extracting and exposing a disease-specific cellular biomarker (such as a disease-specific protein, a disease-specific antibody, a disease-specific antigen, a disease-specific enzyme, a disease-specific peptide, a disease-specific nucleic acid, a disease-specific carbohydrate, a disease-specific hormone, a disease-specific lipid, a disease-specific substrate, and/or a disease-specific metabolite) and the extraction liquid is tested for the presence of the disease-specific cellular biomarker at testing step 1806 by testing the extraction liquid for the presence of the disease-specific cellular biomarker.

For some applications, preparing the extraction liquid at extraction step 1804 comprises extracting, while the animal cells are in contact with at least one of the one or more filters, the cellular components of the animal cells separated from the non-centrifuged oronasopharyngeal fluid sample, for example using any of the testing devices described herein. For other applications, the animal cells are removed from at least one of the one or more filters before the extraction liquid is prepared at extraction step 1804, for example as described hereinabove with reference to FIGS. 1E-F or FIG. 9.

For some applications, preparing the extraction liquid at extraction step 1804 comprises removing and transferring the animal cells and at least a portion of at least one or more of the one or more filters from a testing device after the oronasopharyngeal fluid has passed through the one or more filters, and extracting the cellular components of the animal cells separated from the non-centrifuged oronasopharyngeal fluid sample outside of the testing device, for example, in a test tube, for example as described hereinabove with reference to FIGS. 1E-F. For other applications, preparing the extraction liquid at extraction step 1804 comprises, after the oronasopharyngeal fluid has passed through the one or more filters, removing and transferring at least a portion of at least one or more of the one or more filters into a transport liquid (as described hereinbelow), and thereafter, preparing an extraction liquid by removing and transferring at least a portion of the transport liquid and extracting the cellular components of the animal cells separated from the non-centrifuged oronasopharyngeal fluid sample by extracting the cellular components of the animal cells contained within the at least a portion of the transport liquid, for example in a test tube, such as a PCR tube.

For some applications, the extraction liquid is tested at testing step 1806 while the extraction liquid is in contact with at least one of the one or more porous filters, for example as described hereinabove with reference to FIGS. 1H, 3C, 4H, 5B, 6B, or 7B. For other applications, at least a portion of the extraction liquid is removed from contact with the one or more porous filters before testing at testing step 1806, for example as described hereinabove with reference to FIGS. 1E-F or FIG. 9.

For still other applications, the testing at testing step 1806 comprises applying a transport liquid to at least one of the one or more porous filters to produce a mixture of the extraction liquid and the transport liquid, removing a portion of the mixture from the one or more porous filters, and testing the removed portion of the mixture the presence of the particulate. For other applications, the testing at testing step 1806 comprises removing and transferring at least a portion of the extraction liquid, such as with at least a portion of one of the one or more porous filters, into a transport liquid to produce a mixture of the extraction liquid and the transport liquid, and testing at least a portion of the mixture the presence of the particulate.

For example, the transport liquid may include one or more components selected from the group consisting of:
one or more buffers (e.g., phosphate buffered saline (PBS), Dulbecco's PBS (DPBS), Tris-EDTA buffer solution, Tris-acetate-EDTA buffer solution, and/or Tris-borate-EDTA buffer solution);
one or more antimicrobial agents (e.g., gentamycin and/or amphotericin B);
one or more reducing agents (e.g., dithiothreitol (DTT), ascorbic acid (vitamin C), and/or Cysteine);
one or more protein protectants (e.g., bovine serum albumin and/or heat-inactivated fetal bovine serum);
one or more protease inhibitors (e.g., beta-caproic acid and/or phenylmethylsulfonyl fluoride (PMSF);
one or more nuclease inhibitors (e.g., EDTA); and/or
a detergent (e.g., polysorbate 20 (tween 20), polysorbate 80 (tween 80), and/or Triton X-100).

For example, the transport liquid may comprise Copan Universal Transport Medium (UTM™) (Copan Italia SPA, Brescia, Italy), Hanks' Balanced Salt Solution (Merck KGaA, Darmstadt, Germany), and/or Stuart Transport Media (HiMedia Laboratories Pvt. Ltd., Mumbai, India).

For some applications, at testing step 1806, the extraction liquid is tested for the presence of a virus that may be present in the oral cavity and/or nasal cavity, such as an Influenza virus (e.g., one or more viruses selected from the group of viruses consisting of: Influenza A, Influenza B, Influenza C, and Influenza D); a herpes virus (e.g., one or more viruses selected from the group of viruses consisting of: Epstein-Barr virus and Herpes simplex virus); a coronavirus (e.g., one or more viruses selected from the group of viruses consisting of: SARS-CoV-2, MERS-CoV, and SARS-CoV); a Human papillomavirus (HPV); or a respiratory syncytial virus (RSV)).

For some applications, testing the extraction liquid for presence of the virus at testing step 1806 comprises quantifying a level of the virus.

For some applications, at testing step 1806, the extraction liquid is tested for the presence of a cellular biomarker, such as a tumor-specific cellular biomarker (such as a tumor-specific protein, a tumor-specific antibody, a tumor-specific antigen, a tumor-specific enzyme, a tumor-specific peptide, a tumor-specific nucleic acid, a tumor-specific carbohydrate, a tumor-specific hormone, a tumor-specific lipid, a tumor-specific substrate, and/or a tumor-specific metabolite); an inflammatory-specific cellular biomarker (such as an inflammatory-specific protein, an inflammatory-specific antibody, an inflammatory-specific antigen, an inflammatory-specific enzyme, an inflammatory-specific peptide, an inflammatory-specific nucleic acid, an inflammatory-specific carbohydrate, an inflammatory-specific hormone, an inflammatory-specific lipid, an inflammatory-specific substrate, and/or an inflammatory-specific metabolite); and/or a disease-specific cellular biomarker (such as a disease-specific protein, a disease-specific antibody, a disease-specific antigen, a disease-specific enzyme, a disease-specific peptide, a disease-specific nucleic acid, a disease-specific carbohydrate, a disease-specific hormone, a disease-specific lipid, a disease-specific substrate, and/or a disease-specific metabolite).

For some applications, the cellular biomarker does not include nucleic acid.

For some applications, testing the extraction liquid for presence of the cellular biomarker at testing step 1806 comprises quantifying a level of the cellular biomarker, such as testing if a cellular biomarker is present at higher-than-normal levels. For some applications, testing the extraction liquid for presence of the cellular biomarker at testing step 1806 comprises quantifying a characteristic of the cellular biomarker, such as testing for the presence of abnormal telomere length as an indicator of chromosomal instability associated with some cancers.

For some applications, testing the extraction liquid for the presence of the particulate, such as a virus or a cellular biomarker, at testing step 1806 comprises completing ascertaining whether the particulate is present or whether the particulate is present in higher-than-normal levels within five hours (e.g., within two hours, such as within 20 minutes) of beginning passing the non-centrifuged oronasopharyngeal fluid sample through the one or more porous filters. Alternatively or additionally, for some applications, completing ascertaining whether the particulate is present or whether the particulate is present in higher-than-normal levels comprises completing ascertaining whether the particulate is present or whether the particulate is present in higher-than-normal levels at least 5 minutes after and within 20 minutes of beginning passing the non-centrifuged oronasopharyngeal fluid sample through the one or more porous filters.

For some applications, method 1800 comprises, before passing the non-oronasopharyngeal centrifuged fluid sample through the one or more porous filters, collecting the fluid sample from the human or non-human animal, e.g., from one or more nostrils of the human or non-human animal, from a throat of the human or non-human animal, and/or from a mouth of the human or non-human animal.

For some applications:
passing the non-centrifuged oronasopharyngeal fluid sample through the one or more porous filters at filtering step 1802 comprises passing the non-centrifuged oronasopharyngeal fluid sample through the one or more porous filters while the one or more filters are within a testing device, such as any of the testing devices described herein or a conventional filtration device,
extracting the cellular components of the animal cells at extraction step 1804 comprises extracting the cellular components of the animal cells after filtering step 1802 while the animal cells and the one or more filters are within the testing device, for example as shown in FIGS. 1G-H, and
testing the extraction liquid at testing step 1806 comprises testing the extraction liquid while the extraction liquid is within the testing device, for example as shown in FIGS. 1H, 3C, 4H, 5B, 6B, or 7B, or after the extraction liquid has been removed from the testing device.

For some applications, at extraction step 1804, extracting the cellular components of the animal cells comprises lysing the animal cells.

For some applications, at extraction step 1804, extracting the cellular components of the animal cells comprises applying an extraction reagent to the animal cells, such as described hereinabove with reference to FIG. 1E-F or FIGS. 1H, 3C, 4H, 5B, 6B, or 7B. For example, the extraction reagent may include one or more components selected from the group consisting of:
one or more detergents, e.g., one or more detergents selected from the group consisting of: (a) an ionic detergent, such as Sodium deoxycholate, sodium dodecyl sulphate (SDS), cetyltrimethylammonium bromide (CTAB), CHAPS, or CHAPSO; (b) a non-ionic detergent, such as Triton-X-100, Triton-X-114, NP-40, Tween®-20 (polysorbate-20), Tween®-80 (polysorbate 80), Brij®-35 (SERVA Electrophoresis GmbH), or Octyl glucoside; or (c) a chaotropic detergent, such as urea, guanidine, or ethylenediaminetetraacetic acid (EDTA);
one or more hypertonic solutions, such as salt solutions, e.g., one or more salt solutions selected from the group consisting of: Sodium chloride, Potassium chloride, and Sodium orthovanadate;
one or more hypotonic solutions, such as salt solutions, e.g., one or more hypotonic solutions selected from the group consisting of Sodium chloride, Potassium chloride, Sodium orthovanadate, and pure water;
one or more reducing agents, e.g., one or more reducing agents selected from the group consisting of: dithiothreitol (DTT), 2-Mercaptoethanol, Sodium dithionate, Sodium borohydride, Sodium thiosulfate, and Cysteine;
one or more chelating agents, e.g., one or more chelating agents selected from the group consisting of: EDTA, EGTA, and DMPS;
one or more protease inhibitors, e.g., one or more protease inhibitors selected from the group consisting of: PMSF, Aminocaproic acid, Pepstatin, Aprotinin, and Leupeptin;
one or more proteases, e.g., one or more proteases selected from the group consisting of: Proteinase K, Trypsin, Chymotrypsin, and Endoproteinase Glu-C (V8 protease);
one or more buffers, e.g., one or more buffers selected from the group consisting of: Phosphate buffer, Tris buffer, HEPES, MES buffer, Borate buffer, Acetate buffer, and Carbonate buffer;
one or more alkaline lysing agents, e.g., the combination of sodium dodecyl sulfate (SDS) and a strong base such as sodium hydroxide (NaOH); and/or
one or more enzymatic lysing agents, such as lysozyme, lysostaphin, zymolase, cellulose, protease, or glycanase.

Optionally, applying the extraction reagent to the animal cells comprises applying the extraction reagent to at least one of the one or more filters, for example as shown in FIGS. 1G, 3C, 4H, 5B, 6B, or 7B.

For some applications, at extraction step 1804, extracting the cellular components of the animal cells comprises applying sonication to the animal cells. Optionally, applying the sonication to the animal cells comprises applying the sonication to at least one of the one or more filters.

For some applications, at extraction step 1804, extracting the cellular components of the animal cells comprises applying an electrical field to the animal cells. Optionally, applying the electrical field to the animal cells comprises applying the electrical field to at least one of the one or more filters.

For some applications, at extraction step 1804, extracting the cellular components of the animal cells comprises changing a temperature of the animal cells. Optionally, changing the temperature of the animal cells comprises changing a temperature of at least one of the one or more filters. For some applications, changing the temperature of the animal cells comprises heating the animal cells. Optionally, heating the animal cells comprises heating at least one of the one or more filters. For other applications, changing the temperature of the animal cells comprises freezing and thawing the animal cells. Optionally, freezing and thawing the animal cells comprises freezing and thawing at least one of the one or more filters.

For some applications, at extraction step 1804, the cellular components of the animal cells are physically extracted. For example, physically extracting the cellular components of the animal cells may comprise:
mechanically extracting the cellular components of the animal cells,
manually extracting the cellular components of the animal cells,
applying a physical extraction technique to at least one of the one or more filters, such as agitating at least one of the one or more filters; vibrating at least one of the one or more filters; macerating at least one of the filters; applying osmotic shock to the animal cells, e.g., while they are captured by at least one of the one or more filters; and/or generating cavitation (e.g., using ultrasound) of the animal cells, e.g., while they are captured by at least one of the one or more filters (in which case, at testing step 1806, testing the extraction liquid may optionally comprise testing the extraction liquid, including particles of the one or more filters suspended or partially dissolved in the extraction liquid), and/or
tilting at least one of the one or more filters.

For some applications, the non-centrifuged oronasopharyngeal fluid sample is also non-cultured, and, at filtering step 1802, the non-centrifuged and non-cultured oronasopharyngeal fluid sample is passed through the one or more porous filters.

For some applications, the method further comprises, between filtering step 1802 and extraction step 1804, adding, to at least one of the one or more filters, material (liquid and/or solid) from a nasal swab or a nasopharyngeal swab of the human or non-human animal.

For some applications, passing the non-centrifuged oronasopharyngeal fluid sample through the one or more porous filters to separate the epithelial cells from the non-centrifuged oronasopharyngeal fluid sample at filtering step 1802 comprises passing the non-centrifuged oronasopharyngeal fluid sample through the one or more porous filters to separate the epithelial cells and free viral particles from the non-centrifuged oronasopharyngeal fluid, and preparing the extraction liquid by extracting cellular components of the epithelial cells at extraction step 1804 comprises extracting and exposing a viral target from the cellular components of the epithelial cells and from the free viral particles. Optionally, any of the extraction reagents listed above may be used to perform these techniques.

For some applications, passing the non-centrifuged oronasopharyngeal fluid sample through the one or more porous filters to separate the animal cells from the non-centrifuged oronasopharyngeal fluid sample at filtering step 1802 comprises passing the non-centrifuged oronasopharyngeal fluid sample through the one or more porous filters to separate the animal cells from the non-centrifuged oronasopharyngeal fluid, and preparing the extraction liquid by extracting cellular components of the animal cells at extraction step 1804 comprises extracting and exposing a cellular biomarker from the cellular components of the animal cells. Optionally, any of the extraction reagents listed above may be used to perform these techniques.

Reference is still made to FIG. 11 and is additionally made to FIGS. 12A-B, which are schematic and cross-sectional views of a filter chamber 1900, respectively, in accordance with an application of the present invention. Filter chamber 1900 may be an entire testing device or a component of a testing device (e.g., may be integrated into any of the testing devices described herein, or into other testing devices). Any of the testing devices described herein may implement one or more features of filter chamber 1900, such as, for example, the plurality of filters of filter chamber 1900.

Typically, filter chamber 1900 has an inlet 1938 that is coupled downstream of and in fluid communication with a liquid container, such as any of the liquid containers described herein. Alternatively, for some applications, an upstream portion of filter chamber 1900 is shaped so as to define a liquid container (configuration not shown). Further alternatively, no liquid container is provided.

For some applications, a liquid-pressure source is provided, and is configured to push liquid 22, such as oronasopharyngeal fluid, into filter chamber 1900 via inlet 1938. For example, the liquid-pressure source may implement any of the configurations of liquid-pressure source 34 described hereinabove with reference to FIGS. 1A-H. In configurations in which the liquid-pressure source comprises a plunger, inlet 1938 typically has a greater diameter than illustrated in FIGS. 12A-B.

For some applications, filter chamber 1900 is positioned upstream of and in fluid communication with a waste liquid receptacle (not shown), optionally via an outlet 1940 of filter chamber 1900. Alternatively, for some applications, a downstream portion of filter chamber 1900 is shaped so as to define a waste liquid receptacle (configuration not shown). Further alternatively, no waste liquid receptacle is provided.

Filter chamber 1900 comprises two or more filters 1932, which are arranged in series along a fluid flow path within filter chamber 1900, such as exactly two filters (configuration not shown), exactly three filters 1932A, 1932B, and 1932C (as shown), exactly four filters (configuration not shown), or five or more filters (configuration not shown).

Typically, filter chamber 1900 further comprises filter supports 1942 for filters 1932, respectively, in order to mechanically support the filters during filtration. Filter supports 1942 define relatively large openings to allow essentially uninhibited passage of fluid therethrough.

For some applications, one or more of filters 1932 comprise respective mechanical filters, which are configured to filter by sized-based filtration.

For some of these applications, one or more of the mechanical filters are porous. For example, the porous filters may have respective pore sizes of between 0.01 and 20 microns, which may differ or be the same for the different filters. For some of these applications, two of the two or more porous filters 1932 have average absolute or nominal pore sizes, respectively, which are different from each other. For some of these applications, the more upstream filter has an average absolute or nominal pore size greater than that of the more downstream filter.

For example, for trapping viral particles:
the absolute or nominal pore size of the more upstream filter may between 1.2 and 20 microns, which may be appropriate for filtering animal cells, such as epithelial cells and/or leukocytes that may contain a virus and/or a cellular biomarker, while allowing free viral particles to pass in the filtrate, and/or
the absolute or nominal pore size of the more downstream filter may be between 0.01 and 0.3 microns, which may be appropriate for trapping free viral particles.

For example, for trapping bacteria:
the absolute or nominal pore size of the more upstream filter may between 1.2 and 20 microns, which may be appropriate for filtering animal cells, such as epithelial cells and/or leukocytes that may contain a virus and/or a cellular biomarker, while allowing the bacteria to pass in the filtrate, and/or
the absolute or nominal pore size of the more downstream filter may be between 0.4 and 2.0 microns, which may be appropriate for trapping the bacteria.

For example, for trapping animal cells, bacteria, and free viral particles, three or more porous filters may be provided as follows:
the absolute or nominal pore size of a more upstream filter may between 1.2 and 20 microns, which may be appropriate for filtering animal cells, such as animal cells that contain virus and/or cellular biomarkers, while allowing bacteria and free viral particles to pass in the filtrate,
the absolute or nominal pore size of a middle filter may be between 0.4 and 2.0 microns, which may be appropriate for trapping bacteria, such as Streptococcus bacteria (e.g., Group A Streptococcus, Group C Streptococcus, and Group G Streptococcus), and/or
the absolute or nominal pore size of the more downstream filter may be between 0.01 and 0.3 microns, which may be appropriate for trapping free viral particles, such as of a coronavirus (e.g., one or more viruses selected from the group of viruses consisting of: SARS-CoV-2, MERS-CoV, and SARS-CoV) and/or of an Influenza virus (e.g., one or more viruses selected from the group of viruses consisting of: Influenza A, Influenza B, Influenza C, and Influenza D).

For any of these applications, one or more of filters 1932 may comprise the materials described hereinabove with reference to FIGS. 1A-H for filter 32.

For any of these applications, one or more additional more upstream filters, may be provided, such as one or more most-upstream filters, for prefiltration to increase the efficiency and flow rate of fluid passing through the one or more downstream filters.

For example, for trapping animal cells, bacteria, and free viral particles, four filters may be provided as follows:
the most-upstream filter may have a pore size between 1.2 and 20 microns, such as a borosilicate glass fiber filter with a nominal pore size of 1 and 3 microns, such as 1.2 microns (e.g., Grade C Filter, Sterlitech, Kent, Wash.), to capture animal cells, such as animal cells containing cellular biomarkers;
the next-most-upstream filter may have a pore size between 0.4 and 2 microns, such as a polyethersulfone (PES) filter with an absolute pore size of between 0.4 and 2 microns, such as 0.8 microns (e.g., Polyethersulfone (PES) Membrane Filter 0.8 micron, Sterlitech, Kent, Wash.), to capture bacteria;
the next-most-upstream filter may have a pore size of between 0.2 and 0.5 microns, such as a polyethersulfone (PES) filter with an absolute pore size of between 0.2 and 0.5 microns, such as 0.45 microns (e.g., Polyethersulfone (PES) Membrane Filter 0.45 micron, Sterlitech, Kent, Wash.), to act as a prefilter to improve flow rate; and
the most-downstream filter may have a pore size of between 0.01 and 0.3 microns, such as a polyethersulfone (PES) filter with an absolute pore size of between 0.01 and 0.1 microns, such as 0.03 microns (e.g., Polyethersulfone (PES) Membrane Filter 0.3 micron, Sterlitech, Kent, Wash.), to capture free viral particles.

For example, for trapping animal cells, bacteria, and free viral particles, five filters may be provided as follows:
the most-upstream filter may comprise a filter with a nominal pore size of between 50 and 250 microns (e.g., between 50 and 200 microns, such as between 50 and 150 microns, e.g., between 100 and 150 microns), to act as a prefilter to improve flow rate; for example, the filter may comprise a polymer, such as polyester;
the next-most-upstream filter may have a pore size between 1.2 and 20 microns, such as a polycarbonate filter with an absolute pore size of between 5 and 15 microns, such as 10 microns (e.g., Polycarbonate (PCTE) Membrane Filter 10 micron, Sterlitech, Kent, Wash.), to capture animal cells, such as animal cells containing cellular biomarkers;
the next-most-upstream filter may have a pore size between 0.4 and 2 microns, such as a borosilicate glass fiber filter with a nominal pore size of between 1 and 2 microns, such as 1.2 microns (e.g., Grade C Filter, Sterlitech, Kent, Wash.), to capture bacteria, such as Streptococcus bacteria;
the next-most-upstream filter may serve as a prefilter having a pore size between 0.2 and 1 microns, such as a polyethersulfone (PES) filter with an absolute pore size of between 0.2 and 1 microns, such as 0.8 microns (e.g., Polyethersulfone (PES) Membrane Filter 0.8 micron, Sterlitech, Kent, Wash.), to act as a prefilter to improve flow rate; and
the most-downstream filter may have a pore size of between 0.01 and 0.3 microns, such as a polyethersulfone (PES) filter with an absolute pore size of between 0.01 and 0.1 microns, such as 0.03 microns (e.g., Polyethersulfone (PES) Membrane Filter 0.3 micron, Sterlitech, Kent, Wash.), to capture free viral particles.

Alternatively or additionally, for some of these applications, one or more of the mechanical filters comprise respective depth filters.

Alternatively or additionally, one or more of filters 1932 comprise fixed antibodies configured to capture particles by affinity-based filtration.

Reference is again made to FIGS. 12A-B. For some applications, a distance D between at least one pair of longitudinally-adjacent filters is at least 0.5 mm, and, optionally no more than 3 cm, so as to define a space 1936 between the two longitudinally-adjacent filters. By way of example and not limitation, distance D is labeled between longitudinally-adjacent first porous filter 1932A and second porous filter 1932B.

For some applications, providing this space between the longitudinally-adjacent filters allows for the two or more filters 1932 to be more easily separated, such as to prepare two or more separate extraction liquids, such as to test the two or more separate extraction liquids separately from each other. For some applications, a wall 1934 of filter chamber 1900 is shaped so as to define one or more breakpoints located along space 1936 provided between the two of the two or more filters 1932, such that the filter chamber 1900 can be broken to access the surface of one or more of the two or more filters 1932, such as by twisting or bending the filter chamber 1900.

Reference is still made to FIGS. 11 and 12A-B. For some applications, at filtering step 1802, passing the non-centrifuged oronasopharyngeal fluid sample through the porous filter comprises passing the non-centrifuged oronasopharyngeal fluid sample in series through first and second porous filters 1932 having the first and the second average absolute pore sizes, respectively, described above. For some applications, the extraction liquid is tested at testing step 1806 while the extraction liquid is in contact with both first and second porous filters 1932.

For some applications, passing the non-centrifuged oronasopharyngeal fluid sample through first and second porous filters 1932 to separate the epithelial cells from the non-centrifuged oronasopharyngeal fluid sample at filtering step 1802 comprises passing the non-centrifuged oronasopharyngeal fluid sample through first and second porous filters 1932 to separate the epithelial cells and free viral particles from the non-centrifuged oronasopharyngeal fluid, and preparing the extraction liquid by extracting cellular components of the epithelial cells at extraction step 1804 comprises extracting and exposing a viral target from the cellular components of the epithelial cells and from the free viral particles.

For some applications, at filtering step 1802:
the first average absolute pore size causes first porous filter 1932 primarily to separate the epithelial cells from the non-centrifuged oronasopharyngeal fluid sample, while allowing a filtrate to pass through the first porous filter, the filtrate including free viral particles, and
the second average absolute pore size causes second porous filter 1932 to separate the free viral particles from the filtrate.

The first average absolute pore size may additionally cause first porous filter 1932 to separate a portion of the free viral particles from the non-centrifuged oronasopharyngeal fluid sample. For example, some free viral particles may adhere to the first porous filter 1932, such as to epithelial cells separated by the first porous filter 1932, or may be contained within mucous, and thus may not pass through the first porous filter 1932.

Optionally, any of the extraction reagents listed above may be used to perform these techniques.

For some applications, preparing the extraction liquid at extraction step 1804 comprises extracting, while the animal cells are in contact with first porous filter 1932, the cellular components of the animal cells separated from the non-centrifuged oronasopharyngeal fluid sample. Alternatively or additionally, for some applications, the extraction liquid is tested at testing step 1806 while the extraction liquid is in contact with first porous filter 1932, and second porous filter 1932 is additionally tested for the presence of free viral particles of the virus. Optionally, a portion of the extraction liquid passes through first porous filter 1932 at extraction step 1804, and the extraction liquid is tested at testing 1806 while the extraction liquid is in contact with both first and second porous filters 1932.

For some applications, the one or more porous filters are one or more cell-trapping filters, and testing the extraction liquid for the presence of the virus comprises:
passing the extraction liquid through a virus-trapping filter to trap at least a portion of the free viral particles; and
thereafter, testing for the presence of the free viral particles trapped by the virus-trapping filter.

For some of these applications, the virus-trapping filter comprises a mechanical filter, and passing the extraction liquid through the virus-trapping filter comprises passing the extraction liquid through the virus mechanical filter to trap the at least a portion of the free viral particles by size-based filtration. For some applications, the virus mechanical filter comprises a depth filter. For some applications, the virus-trapping filter comprises fixed antibodies configured to capture the free viral particles by affinity-based filtration.

Reference is again made to FIGS. 11 and 12A-B. For some applications, the techniques described with reference to these figures are used to test for the presence of a non-viral pathogen, such as bacteria, e.g., Streptococcus bacteria (e.g., Group A Streptococcus, Group C Streptococcus, and Group G Streptococcus), Bordetella pertussis, Chlamydia trachomatis, or a mycoplasma.

Size-Based Filtration Capture of Epithelial Cells from Gargled Fluid and Nasal Wash Followed by Influenza A+B Viral Testing by Rapid Antigen Detection Immunoassay: Two Case Studies

### 1. First Case Study: Gargled Fluid

### 1.1. Materials & Methods

Female, 8 years old, presented to physician with flu-like symptoms on December 23, 2019. Standard nasal swab was performed and tested using McKesson Consult™ Immunoassay Influenza A+B Test kit (Ref: 181-36025. Lot: 448LZ1. Exp: 2020.11.30) as a control. Patient was asked to gargle 11 mL of phosphate buffered saline (PBS) to produce gargled fluid, but patient was not able to gargle properly and coughed out fluid quickly with no gargling sound. The gargled fluid was filtered to capture epithelial cells using a Grade C 25mm Glass Microfiber Filter (Sterilitech Corporation. Ref: C2500. Lot: 510563) that has a nominal pore size of 1.2 microns. After filtration, filter was transferred into a tube containing McKesson Consult™ Immunoassay Influenza A+B Test kit lysis buffer and was macerated using a stirring rod for —45 seconds to prepare an extraction liquid by extracting and exposing the viral antigen from the epithelial cells. Filter was maintained in the lysis buffer for ˜3 minutes and then transferred into the McKesson Consult™ Immunoassay Influenza A+B Test kit cassette. Additional McKesson Consult™ Immunoassay Influenza A+B Test kit lysis buffer was added to the filter in the cassette and results were read 10 minutes after cassette test initiation and 15 minutes after cassette test initiation.

### 1.2. Results

| | Results at 10 minutes | Results at 15 minutes |
|---|---|---|
| Control: Nasal Swab | Positive | Positive |
| Gargled Fluid Filtration | Positive | Positive |

### 2. Second Case Study: Nasal Wash

### 2.1. Materials & Methods

Male, 18 years old, presented to physician with flu-like symptoms on February 16, 2020. Standard nasal swab was performed and tested using Quidel QuickVue® Influenza A+B Test kit (Ref: 20183. Lot: 137286. Exp: 2019.09.30) as a control. Physician performed a nasal wash by inserting 2.5 mL of phosphate buffered saline (PBS) into each nostril. Approximately 4 mL of nasal wash was collected from the patient and was filtered to capture epithelial cells using a Grade C 25mm Glass Microfiber Filter (Sterilitech

Corporation. Ref: C2500. Lot: 510563) that has a nominal pore size of 1.2 microns. After filtration, filter was transferred into a tube containing 31 4 of Polysorbate 20 (Alfa Aesar. Ref: L15029. Lot: 10203660) and 280 4 of Quidel QuickVue® Influenza A+B Test kit lysis buffer and was macerated using a stirring rod for —45 seconds to prepare an extraction liquid by extracting and exposing the viral antigen from the epithelial cells. Filter was maintained in the lysis buffer for —3 minutes and then Quidel QuickVue® Influenza A+B Test kit lateral-flow dipstick was added. Results were read 10 minutes after dipstick test initiation and 15 minutes after dipstick test initiation.

### 2.2. Results

| | Results at 10 minutes | Results at 15 minutes |
|---|---|---|
| Control: Nasal Swab | Positive | Positive |
| Nasal Wash Filtration | Negative | Positive |

### 3. Conclusions

The aforementioned case studies support the feasibility of detecting the presence of a virus by filtering oronasal fluid to capture epithelial cells followed by testing the filter for the presence of the virus within the epithelial cells by using an immunoassay.

In an embodiment, the techniques and apparatus described herein are combined with techniques and apparatus described in one or more of the following patent applications, which are assigned to the assignee of the present application and are incorporated herein by reference:
PCT Publication WO 2018/158768 to Fruchter et al.;
U.S. Provisional Application 62/727,208, filed Sep. 5, 2018;
U.S. Provisional Application 62/727,268, filed Sep. 5, 2018;
PCT Publication WO 2020/049566 to Fruchter et al.;
PCT Publication WO 2020/049569 to Fruchter et al.;
U.S. Provisional Application 62/896,295, filed Sep. 5, 2019;
U.S. Provisional Application 62/988,259, filed Mar. 11, 2020;
U.S. Provisional Application 62/988,145, filed Mar. 11, 2020;

International Application PCT/IL2020/050957, filed Sep. 3, 2020; and/or an international application filed on even date herewith, entitled, “Testing devices.”

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method for testing a non-centrifuged oronasopharyngeal fluid sample taken from a human or non-human animal for the presence of a virus, the method comprising:
passing the non-centrifuged oronasopharyngeal fluid sample through one or more porous filters to separate epithelial cells from the non-centrifuged oronasopharyngeal fluid sample by size-based filtration;
thereafter, preparing an extraction liquid by extracting cellular components of the epithelial cells separated from the non-centrifuged oronasopharyngeal fluid sample; and
thereafter, testing the extraction liquid for the presence of the virus.

2. The method according to claim 1, wherein testing the extraction liquid comprises testing the extraction liquid while the extraction liquid is in contact with at least one of the one or more porous filters.

3. The method according to claim 1, wherein testing the extraction liquid for the presence of the virus comprises removing at least a portion of the extraction liquid from contact with the one or more porous filters, and testing at least a portion of the removed extraction liquid.

4. The method according to claim 1, wherein testing the extraction liquid for the presence of the virus comprises applying a transport liquid to at least one of the one or more porous filters to produce a mixture of the extraction liquid and the transport liquid, removing a portion of the mixture from the at least one of the one or more porous filters, and testing the removed mixture for the presence of the virus.

5. The method according to claim 1, wherein preparing the extraction liquid comprises extracting, while the epithelial cells are in contact with at least one of the one or more porous filters, the cellular components of the epithelial cells separated from the non-centrifuged oronasopharyngeal fluid sample.

6. The method according to claim 1, wherein testing for presence of the virus comprises quantifying a level of the virus.

7. The method according to claim 1, wherein filtering the non-centrifuged oronasopharyngeal fluid sample comprises filtering between 0.5 and 50 cc of the non-centrifuged oronasopharyngeal fluid sample.

8. The method according to claim 7, wherein filtering the non-centrifuged oronasopharyngeal fluid sample comprises filtering between 4 and 25 cc of the non-centrifuged oronasopharyngeal fluid sample.

9. The method according to claim 1, wherein the non-centrifuged oronasopharyngeal fluid sample is also non-cultured, and wherein passing the non-centrifuged oronasopharyngeal fluid sample through the one or more porous filters comprises passing the non-centrifuged and non-cultured oronasopharyngeal fluid sample through the one or more porous filters.

10. The method according to claim 1, wherein at least one of the one or more porous filters has an average absolute pore size of between 0.01 and 20 microns.

11. The method according to claim 10, wherein the average absolute pore size is between 0.1 and 20 microns.

12. The method according to claim 10, wherein the average absolute pore size is between 0.45 and 9.0 microns.

13. The method according to claim 10, wherein the average absolute pore size is between 1.2 and 20 microns.

14. The method according to claim 13, wherein the average absolute pore size is between 2.0 and 20 microns.

15. The method according to claim 14, wherein the average absolute pore size is between 5.0 and 20 microns.

16. The method according to claim 12, wherein the average absolute pore size is between 0.45 and 2.0 microns.

17. The method according to claim 1, wherein at least one of the one or more porous filters has an average nominal pore size of between 0.01 and 20 microns.

18. The method according to claim 17, wherein the average nominal pore size is between 0.1 and 20 microns.

19. The method according to claim 18, wherein the average nominal pore size is between 0.45 and 9.0 microns.

20. The method according to claim 17, wherein the average nominal pore size is between 1.2 and 20 microns.

21. The method according to claim 20, wherein the average nominal pore size is between 1.2 and 20 microns.

22. The method according to claim 21, wherein the average nominal pore size is between 5.0 and 20 microns.

23. The method according to claim 19, wherein the average nominal pore size is between 0.45 and 2.0 microns.

24. The method according to claim 1, wherein extracting the cellular components of the epithelial cells comprises lysing the epithelial cells.

25. The method according to claim 1, further comprising, after passing the non-centrifuged oronasopharyngeal fluid sample through the one or more porous filters and before preparing the extraction liquid, adding, to at least one of the one or more porous filters, material from a nasal swab or a nasopharyngeal swab of the human or non-human animal.

26. The method according to any one of claims 1-25,
wherein preparing the extraction liquid comprises extracting and exposing a viral target, and
wherein testing the extraction liquid for the presence of the virus comprises testing the extraction liquid for the presence of the viral target.

27. The method according to claim 26,
wherein extracting and exposing the viral target comprises extracting and exposing a viral antigen, and
wherein testing the extraction liquid for the presence of the viral target comprises testing the extraction liquid for the presence of the viral antigen.

28. The method according to claim 26,
wherein extracting and exposing the viral target comprises extracting and exposing a viral nucleic acid, and
wherein testing the extraction liquid for the presence of the viral target comprises testing the extraction liquid for the presence of the viral nucleic acid.

29. The method according to claim 26,
wherein extracting and exposing the viral target comprises extracting and exposing a viral nucleoprotein, and
wherein testing the extraction liquid for the presence of the viral target comprises testing the extraction liquid for the presence of the viral nucleoprotein.

30. The method according to any one of claims 1-25,
wherein passing the non-centrifuged oronasopharyngeal fluid sample through the one or more porous filters to separate the epithelial cells from the non-centrifuged oronasopharyngeal fluid sample comprises passing the non-centrifuged oronasopharyngeal fluid sample through the one or more porous filters to separate the epithelial cells and free viral particles from the non-centrifuged oronasopharyngeal fluid, and
wherein preparing the extraction liquid by extracting cellular components of the epithelial cells comprises extracting and exposing a viral target from the cellular components of the epithelial cells and from the free viral particles.

31. The method according to any one of claims 1-25, wherein the non-centrifuged oronasopharyngeal fluid sample taken from the human or non-human animal is one or more non-centrifuged oronasopharyngeal fluid samples taken from one or more of oronasopharyngeal cavities of the human or non-human animal.

32. The method according to claim 31, wherein the non-centrifuged oronasopharyngeal fluid sample includes a non-centrifuged nasal wash taken from the human or non-human animal.

33. The method according to claim 31, wherein the non-centrifuged oronasopharyngeal fluid sample includes non-centrifuged nasal aspirate taken from the human or non-human animal.

34. The method according to claim 31, wherein the non-centrifuged oronasopharyngeal fluid sample includes non-centrifuged gargled fluid taken from the human or non-human animal.

35. The method according to claim 31, wherein the non-centrifuged oronasopharyngeal fluid sample includes a non-centrifuged oral wash taken from the human or non-human animal.

36. The method according to claim 31, wherein the non-centrifuged oronasopharyngeal fluid sample includes saliva taken from the human or non-human animal.

37. The method according to claim 31, wherein the non-centrifuged oronasopharyngeal fluid sample includes (a) material from (i) a nasal swab or (ii) a nasopharyngeal swab mixed with (b) (i) non-centrifuged gargled fluid taken from the human or non-human animal or (ii) nasal wash taken from the human or non-human animal.

38. The method according to any one of claims 1-25, wherein testing the extraction liquid for the presence of the virus comprises testing the extraction liquid for the presence of a virus that may be present in an upper respiratory tract.

39. The method according to claim 38, wherein the virus that may be present in the upper respiratory tract is one or more viruses selected from the group of viruses consisting of: Epstein-Ban virus (EBV), Herpes simplex virus (HSV), a Human papillomavirus (HPV), an Influenza virus, a coronavirus, and a respiratory syncytial virus (RSV).

40. The method according to claim 39, wherein the virus is the Influenza virus.

41. The method according to claim 40, wherein the Influenza virus is one or more viruses selected from the group of viruses consisting of: Influenza A, Influenza B, Influenza C, and Influenza D.

42. The method according to claim 39, wherein the virus is the coronavirus.

43. The method according to claim 42, wherein the coronavirus is one or more viruses selected from the group of viruses consisting of: SARS-CoV-2, MERS-CoV, and SARS-CoV.

44. The method according to claim 39, wherein the virus is the respiratory syncytial virus (RSV).

45. The method according to any one of claims 1-25, wherein testing the extraction liquid comprises performing an immunoassay on the extraction liquid.

46. The method according to claim 45, wherein performing the immunoassay comprises performing a lateral flow immunoassay on the extraction liquid.

47. The method according to claim 45, wherein performing the immunoassay comprises performing an enzyme-linked immunosorbent assay (ELISA) on the extraction liquid.

48. The method according to any one of claims 1-25, wherein testing the extraction liquid comprises performing a molecular-based assay on the extraction liquid.

49. The method according to claim 48, wherein performing the molecular-based assay on the extraction liquid comprises performing nucleic acid hybridization.

50. The method according to claim 48, wherein performing the molecular-based assay on the extraction liquid comprises performing nucleic acid amplification.

51. The method according to claim 50, wherein the nucleic acid amplification is selected from the group consisting of: polymerase chain reaction (PCR) amplification, real-time quantitative PCR (qPCR) amplification, reverse transcriptase PCR (RT-PCR) amplification, and isothermal amplification.

52. The method according to any one of claims 1-25, wherein extracting the cellular components of the epithelial cells comprises applying an extraction reagent to the epithelial cells.

53. The method according to claim 52, wherein the extraction reagent includes one or more components selected from the group consisting of: one or more detergents, one or more salt solutions, one or more hypertonic solutions, one or more hypotonic solutions, one or more reducing agents, one or more chelating agents, one or more protease inhibitors, one or more proteases, one or more alkaline lysing agents, one or more enzymatic lysing agents, and one or more buffers.

54. The method according to claim 53, wherein the extraction reagent includes one or more detergents selected from the group consisting of: an ionic detergent, a non-ionic detergent, and a chaotropic detergent.

55. The method according to claim 54, wherein the extraction reagent includes one or more ionic detergents selected from the group consisting of: Sodium deoxycholate, sodium dodecyl sulphate (SDS), cetyltrimethylammonium bromide (CTAB), CHAPS, and CHAPSO.

56. The method according to claim 54, wherein the extraction reagent includes one or more non-ionic detergents selected from the group consisting of: Triton-X-100, Triton-X-114, NP-40, polysorbate-20, polysorbate 80, and Octyl glucoside.

57. The method according to claim 54, wherein the extraction reagent includes one or more chaotropic detergents selected from the group consisting of: urea, guanidine, and ethylenediaminetetraacetic acid (EDTA).

58. The method according to claim 53, wherein the extraction reagent includes one or more hypertonic solutions selected from the group consisting of: Sodium chloride, Potassium chloride, and Sodium orthovanadate.

59. The method according to claim 53, wherein the extraction reagent includes one or more hypotonic solutions selected from the group consisting of: Sodium chloride, Potassium chloride, Sodium orthovanadate, and pure water.

60. The method according to claim 53, wherein the extraction reagent includes one or more reducing agents selected from the group consisting of: dithiothreitol (DTT), 2-Mercaptoethanol, Sodium dithionate, Sodium borohydride, Sodium thiosulfate, and Cysteine.

61. The method according to claim 53, wherein the extraction reagent includes one or more chelating agents selected from the group consisting of: EDTA, EGTA, and DMPS.

62. The method according to claim 53, wherein the extraction reagent includes one or more protease inhibitors selected from the group consisting of: PMSF, Aminocaproic acid, Pepstatin, Aprotinin, and Leupeptin.

63. The method according to claim 53, wherein the extraction reagent includes one or more proteases selected from the group consisting of: Proteinase K, Trypsin, Chymotrypsin, and Endoproteinase Glu-C (V8 protease).

64. The method according to claim 53, wherein the extraction reagent includes one or more buffers selected from the group consisting of: Phosphate buffer, Tris buffer, HEPES, MES buffer, Borate buffer, Acetate buffer, and Carbonate buffer.

65. The method according to claim 52, wherein applying the extraction reagent to the epithelial cells comprises applying the extraction reagent to at least one of the one or more porous filters.

66. The method according to any one of claims 1-25, wherein extracting the cellular components of the epithelial cells comprises applying sonication to the epithelial cells.

67. The method according to claim 66, wherein applying the sonication to the epithelial cells comprises applying the sonication to at least one of the one or more porous filters.

68. The method according to any one of claims 1-25, wherein extracting the cellular components of the epithelial cells comprises applying an electrical field to the epithelial cells.

69. The method according to claim 68, wherein applying the electrical field to the epithelial cells comprises applying the electrical field to at least one of the one or more porous filters.

70. The method according to any one of claims 1-25, wherein extracting the cellular components of the epithelial cells comprises changing a temperature of the epithelial cells.

71. The method according to claim 70, wherein changing the temperature of the epithelial cells comprises changing a temperature of at least one of the one or more porous filters.

72. The method according to claim 70, wherein changing the temperature of the epithelial cells comprises heating the epithelial cells.

73. The method according to claim 72, wherein heating the epithelial cells comprises heating at least one of the one or more porous filters.

74. The method according to claim 70, wherein changing the temperature of the epithelial cells comprises freezing and thawing the epithelial cells.

75. The method according to claim 72, wherein freezing and thawing the epithelial cells comprises freezing and thawing at least one of the one or more porous filters.

76. The method according to any one of claims 1-25, wherein extracting the cellular components of the epithelial cells comprises physically extracting the cellular components of the epithelial cells.

77. The method according to claim 76, wherein physically extracting the cellular components of the epithelial cells comprises mechanically extracting the cellular components of the epithelial cells.

78. The method according to claim 76, wherein physically extracting the cellular components of the epithelial cells comprises manually extracting the cellular components of the epithelial cells.

79. The method according to claim 76, wherein physically extracting the cellular components of the epithelial cells comprises applying a physical extraction technique to at least one of the one or more porous filters.

80. The method according to claim 79, wherein applying the physical extraction technique to the at least one of the one or more porous filters comprises agitating the at least one of the one or more porous filters.

81. The method according to claim 79, wherein applying the physical extraction technique to the at least one of the one or more porous filters comprises vibrating the at least one of the one or more porous filters.

82. The method according to claim 79, wherein applying the physical extraction technique to the at least one of the one or more porous filters comprises macerating the at least one of the one or more porous filters.

83. The method according to claim 82, wherein testing the extraction liquid comprises testing the extraction liquid, including particles of the at least one of the one or more porous filters suspended or partially dissolved in the extraction liquid.

84. The method according to claim 79, wherein applying the physical extraction technique to the at least one of the one or more porous filters comprises tilting the at least one of the one or more porous filters.

85. The method according to any one of claims 1-25, wherein passing the non-centrifuged oronasopharyngeal fluid sample through the one or more porous filters comprises passing the non-centrifuged oronasopharyngeal fluid sample in series through first and second porous filters having first and second average absolute pore sizes, respectively, the first average absolute pore size greater than the second absolute pore size.

86. The method according to claim 85, wherein the first average absolute pore size is between 1.2 and 20 microns, and the second average absolute pore size is between 0.01 and 0.3 microns.

87. The method according to claim 85, passing the non-centrifuged oronasopharyngeal fluid sample in series through the first and the second porous filters comprises:
passing the non-centrifuged oronasopharyngeal fluid sample through the first porous filter to separate the epithelial cells from the non-centrifuged oronasopharyngeal fluid sample by size-based filtration, and to allow a filtrate to pass through the first porous filter, the filtrate including free viral particles; and
passing the filtrate through the second porous filter to separate the free viral particles from the filtrate by size-based filtration.

88. The method according to claim 87, wherein testing the extraction liquid comprises testing the extraction liquid while the extraction liquid is in contact with at least the second porous filter.

89. The method according to claim 88, wherein testing the extraction liquid comprises testing the extraction liquid while the extraction liquid is in contact with at least the first and the second porous filters.

90. The method according to claim 88, wherein testing the extraction liquid while the extraction liquid is in contact with at least the first and the second porous filters comprises testing a first extraction liquid while the first extraction liquid is in contact with at least the first porous filter and testing a second extraction liquid while the second extraction liquid is in contact with at least the second porous filter.

91. The method according to any one of claims 1-25,
wherein the one or more porous filters are one or more cell-trapping filters, and
wherein testing the extraction liquid for the presence of the virus comprises:
passing the extraction liquid through a virus-trapping filter to trap at least a portion of free viral particles; and
thereafter, testing for the presence of the free viral particles trapped by the virus-trapping filter.

92. The method according to claim 91, wherein the virus-trapping filter comprises a mechanical filter, and wherein passing the extraction liquid through the virus-trapping filter comprises passing the extraction liquid through the virus mechanical filter to trap the at least a portion of the free viral particles by size-based filtration.

93. The method according to claim 91, wherein the virus-trapping filter comprises fixed antibodies configured to capture the free viral particles by affinity-based filtration.

94. The method according to any one of claims 1-25,
wherein passing the non-centrifuged oronasopharyngeal fluid sample through the one or more porous filters comprises passing the non-centrifuged oronasopharyngeal fluid sample through the one or more porous filters while the one or more porous filters are within a testing device, and
wherein extracting the cellular components of the epithelial cells comprises extracting the cellular components of the epithelial cells while the epithelial cells and the one or more porous filters are within the testing device.

95. The method according to claim 94, wherein extracting the cellular components of the epithelial cells comprises applying at least one extraction agent to the one or more porous filters while the one or more porous filters are within the testing device.

96. The method according to claim 94, wherein testing the extraction liquid comprises testing the extraction liquid while the extraction liquid is within the testing device.

97. The method according to claim 96, wherein testing the extraction liquid for the presence of the virus comprises inserting a test strip into the testing device.

98. The method according to claim 94, wherein testing the extraction liquid comprises removing the extraction liquid from the testing device and thereafter testing the extraction liquid.

99. The method according to claim 98, wherein testing the extraction liquid comprises removing the extraction liquid and at least a portion of the filter from the testing device and thereafter testing the extraction liquid and the at least a portion of the filter.

100. The method according to any one of claims 1-25,
wherein passing the non-centrifuged oronasopharyngeal fluid sample through the one or more porous filters comprises passing the non-centrifuged oronasopharyngeal fluid sample through the one or more porous filters while the one or more porous filters are within a testing device, and
wherein extracting the cellular components of the epithelial cells comprises removing at least a portion of the one or more porous filters from the testing device and thereafter extracting the cellular components of the epithelial cells while the epithelial cells and the at least a portion of the one or more porous filters are outside the testing device.

101. The method according to claim 100, wherein testing the extraction liquid for the presence of the virus comprises performing a detection test on the extraction liquid outside the testing device.

102. The method according to claim 101, wherein testing the extraction liquid for the presence of the virus comprises performing a nucleic acid amplification detection test on the extraction liquid outside the testing device.

103. The method according to any one of claims 1-25, wherein testing the extraction liquid for the presence of the virus comprises completing ascertaining whether the virus is present within five hours of beginning passing the non-centrifuged oronasopharyngeal fluid sample through the one or more porous filters.

104. The method according to claim 103, wherein completing ascertaining whether the virus is present comprises completing ascertaining whether the virus is present within two hours of beginning passing the non-centrifuged oronasopharyngeal fluid sample through the one or more porous filters.

105. The method according to claim 103, wherein completing ascertaining whether the virus is present comprises completing ascertaining whether the virus is present at least 5 minutes after and within 20 minutes of beginning passing the non-centrifuged oronasopharyngeal fluid sample through the one or more porous filters.

106. The method according to claim 105, wherein completing ascertaining whether the virus is present comprises completing ascertaining whether the virus is present at least 8 minutes after and within 20 minutes of beginning passing the non-centrifuged oronasopharyngeal fluid sample through the one or more porous filters.

107. The method according to claim 106, wherein completing ascertaining whether the virus is present comprises completing ascertaining whether the virus is present at least 12 minutes after and within 20 minutes of beginning passing the non-centrifuged oronasopharyngeal fluid sample through the one or more porous filters.

108. The method according to claim 107, wherein completing ascertaining whether the virus is present comprises completing ascertaining whether the virus is present at least 15 minutes after and within 20 minutes of beginning passing the non-centrifuged oronasopharyngeal fluid sample through the one or more porous filters.

109. The method according to any one of claims 1-25, further comprising, before passing the non-centrifuged oronasopharyngeal fluid sample through the one or more porous filters, collecting the fluid sample from the human or non-human animal.

110. A method for testing a non-centrifuged oronasopharyngeal fluid sample taken from a human or non-human animal for the presence of a cellular biomarker, the method comprising:
passing the non-centrifuged oronasopharyngeal fluid sample through one or more porous filters to separate animal cells from the non-centrifuged oronasopharyngeal fluid sample by size-based filtration;
thereafter, preparing an extraction liquid by extracting cellular components of the animal cells separated from the non-centrifuged oronasopharyngeal fluid sample; and
thereafter, testing the extraction liquid for the presence of the cellular biomarker.

111. A method for testing a non-centrifuged oronasopharyngeal fluid sample taken from a human or non-human animal for the presence of at least cellular components of animal cells, bacteria, and free viral particles, the method comprising:
passing the non-centrifuged oronasopharyngeal fluid sample through a cell-trapping porous filter to separate the animal cells from the non-centrifuged oronasopharyngeal fluid sample by size-based filtration and to allow a first filtrate to pass through the cell-trapping porous filter, the first filtrate potentially including the bacteria and the free viral particles;
passing the first filtrate through a bacteria-trapping porous filter to separate the bacteria from the non-centrifuged oronasopharyngeal fluid sample by size-based filtration and to allow a second filtrate to pass through the bacteria porous filter, the second filtrate potentially including the free viral particles;
passing the second filtrate through a virus-trapping porous filter to separate the free viral particles from the non-centrifuged oronasopharyngeal fluid sample;
thereafter, preparing at least:
(a) a first extraction liquid by extracting the cellular components of the animal cells separated from the non-centrifuged oronasopharyngeal fluid sample by the cell-trapping porous filter,
(b) a second extraction liquid by extracting a bacterial target from the bacteria separated from first filtrate by the bacteria-trapping porous filter, and
(c) a third extraction liquid by extracting a viral target from the free viral particles separated from the second filtrate by the virus-trapping porous filter; and
thereafter:
(a) testing the first extraction liquid,
(b) testing the second extraction liquid for the presence of the bacteria, and
(c) testing the third extraction liquid for the presence of the free viral particles.

112. The method according to claim 111, wherein testing the first extraction liquid comprises testing the first extraction liquid for the presence of a virus.

113. The method according to claim 111, wherein testing the first extraction liquid comprises testing the first extraction liquid for the presence of a cellular biomarker.
